(19) United States
(12) Patent Application Publication
(10) Pub. No.: US 2022/0177894 A1
(43) Pub. Date: Jun. 9, 2022

(54) ANTISENSE OLIGONUCLEOTIDES FOR IMMUNOTHERAPY

(71) Applicant: ProQR Therapeutics II B.V., Leiden (NL)

(72) Inventors: Aliye Seda Yilmaz-Elis, Leiden (NL); Marko Potman, Leiden (NL); Gerardus Johannes Platenburg, Leiden (NL)

(21) Appl. No.: 17/442,913

(22) PCT Filed: Mar. 27, 2020
(86) PCT No.: PCT/EP2020/058828

§ 371 Date: Sep. 24, 2021

(30) Foreign Application Priority Data
Apr. 2, 2019 (GB) 1904647.3
Jun. 12, 2019 (GB) 1908441.7

(51) Int. Cl. C12N 15/113

(52) U.S. Cl. CPC C12N 15/1138; C12N 2310/33; C12N 2310/315; C12N 2310/11

(57) ABSTRACT

The invention relates to antisense oligonucleotides (AON) capable of inducing the skip of at least exon 3 from (human) CD274 pre-mRNA to render a shortened PD-L1 protein, and thereby modulating the function of PD-L1. Preferably, PD-L1 that is produced after the skip of exon 3 from its pre-mRNA is no longer able to traffic to the cell membrane and/or is no longer able to (fully) interact with its receptor PD-1. The result is preferably that the PD-1/PD-L1 pathway is blocked and T cell exhaustion is diminished, prevented or lowered. The AONs of the present invention are particularly useful in immunotherapy and can be applied in the treatment, prevention, and amelioration of (acute or chronic) viral infections, cancer and (auto-) immune disease, especially those disorders in which T cell exhaustion plays a role. The invention relates to AONs, pharmaceutical compositions comprising such AONs, and viral vectors expressing such AONs, that may be used in the treatment of subjects that may benefit from modulation of PD-L1 function.

### FIELD OF THE INVENTION

The invention relates to the field of medicine and relates to the field of immunotherapy, and even more in particular to antisense oligonucleotides (AONs) that are used for modulating the functionality of human programmed death-ligand 1 (PD-L1). More specifically, the invention relates to AONs that induce skipping of one or more exons from human CD274 pre-mRNA that encodes PD-L1.

### BACKGROUND OF THE INVENTION

Hepatitis B virus (HBV) infection is the major cause of inflammatory liver diseases, such as chronic hepatitis, liver cirrhosis and hepatocellular carcinoma. In humans, chronic HBV infection often shows weak or absent virus-specific T-cell reactivity, which is described as the ‘exhaustion’ state characterized by poor effector cytotoxic activity, impaired cytokine production and sustained expression of multiple inhibitory receptors, such as programmed cell death-1 (PD-1, or CD279), lymphocyte activation gene-3 (LAG-3, or CD223), cytotoxic T lymphocyte-associated antigen-4 (CTLA-4, or CD152), Tim-3, CD160, TIGIT, and 2B4 (CD244). As both CD4+ and CD8+ T cells participate in the immune responses against chronic hepatitis virus through distinct manners, compelling evidence has accumulated that shows that the anti-viral function of these exhausted T cells is restored by blocking the interaction between those inhibitory receptors and their respective ligands. T-cell exhaustion plays a major role in (chronic and acute) virus infections, such as those with lymphocytic choriomeningitis virus (LCMV; Kahan and Zajac. 2019, Viruses 11(156)), hepatitis C virus (HCV; Golden-Mason et al. 2007, J Virol 81:9249-9258; Urbani et al. 2006, J Virol 80:11398-11403), human immunodeficiency virus (HIV; Day et al. 2006, Nature 443:350-354), HBV (Boni et al. 2007, J Virol 81:4215-4225), as well as in certain cancers. The functional restoration of HCV- and HIV-specific CD8+ T cells by PD-1 blockade has been verified, whereas it is anticipated that blocking the PD-1 pathway could also be an important immunotherapeutic strategy for the immunological control of tumours in humans, because the interaction between PD-1 and its ligand programmed death-ligand 1 (PD-L1) plays a critical role in T-cell exhaustion (Barber et al. 2006, Nature 439:682-687; Maier et al. 2007, J Immunol 178:2714-2720; Velu et al. 2009, Nature 458:206-210). While it is not expressed on naïve T cells, PD-1 is transiently expressed after activation and functions to down-modulate the anti-viral response (Sharpe and Pauken. 2018, Nat Rev Immunol 18:153-167; Odorizzi et al. 2015, J Exp Med 212:1125-1137). During chronic LCMV infection, for instance, the levels of PD-1 remain elevated on CD8+ T cells as exhaustion sets in (Blackburn et al. 2008, Nat Immunol 10:29-37).

A high level of PD-1 expression is a common feature of T cells during chronic infections including HBV, HCV and HIV infections. PD-1 is also expressed by tumour-reactive T cells during many cancers, and targeting this inhibitory pathway is the basis of major checkpoint blockade approach for cancer therapy (Ahmadzadeh et al. 2009, Blood 114:1537-1544; Baitsch et al. 2011, J Clin Investig 121:2350-2360; Curran et al. 2010, Proc Natl Acad Sci USA 107:4275-4280; Thommen and Schumacher. 2018, Cancer Cell 33:547-562). The blockade of PD-1/PD-L1 interactions increased HBcAg-specific interferon-gamma (IFN-γ) production in intrahepatic T lymphocytes, whereas an anti-PD-1 monoclonal antibody reversed the exhausted phenotype in intrahepatic T lymphocytes and viral persistence to clearance of HBV in vivo (Tzeng et al. 2012, PLos ONE 7(6):e39179).

PD-L1 (also known as cluster of differentiation 274, or CD274, and as B7 homolog 1, or B7-H1) is a 40 kDa type 1 transmembrane protein that appears to act in suppressing the adaptive arm of the immune system during events such as pregnancy, tissue allografts, autoimmune disease, and as indicated above, hepatitis. Several human cancer cells express high levels of PD-L1 and it is known that blocking PD-L1 may reduce the growth of tumours in the presence of immune cells. PD-L1 binds to its receptor PD-1 found on activated T cells, B cells and myeloid cells, to modulate activation or inhibition, by delivering a signal that inhibits TCR-mediated activation of IL-2 production and T cell proliferation. PD-L1 binding to PD-1 also contributes to ligand-induced TCR down-modulation during antigen presentation to naïve T cells, by inducing the up-regulation of the E3 ubiquitin ligase CBL-b. Upregulation of PD-L1 may allow cancers to evade the host immune system.

Many PD-1 and PD-L1 inhibitors have been approved or are in development as immune-oncology and antiviral infection therapies. Since both proteins are expressed on the surface of cells it makes them clear candidates for antibody-based targeting. In general, such inhibitors aim to disrupt the association between the two proteins. Over the last two decades a wide variety of antibodies were tested that would interrupt the interaction between PD-1 and PD-L1, some of which have been formally approved for cancer treatment. Examples of FDA-approved anti-PD-1 antibodies are Pembrolizumab, which has been approved for the treatment of non-small lung cancer and head and neck squamous cell carcinoma, Nivolumab, which is approved for the treatment of squamous cell lung cancer, renal carcinoma and Hodgkin's lymphoma, and Cemiplimab, which is approved for the treatment of cutaneous squamous cell carcinoma. Examples of anti-PD-L1 antibodies are Atezolizumab, which was approved for the treatment of urothelial carcinoma and non-small cell lung cancer, Avelumab, which was approved for the treatment of metastatic merkel-cell carcinoma, and Durvalumab, which was approved for the treatment of urothelial carcinoma and unresectable non-small cell lung cancer.

It has been demonstrated that influenza virus infection of primary airway epithelial cells strongly enhances PD-L1 expression and does so in an alpha interferon receptor (IFNAR) signalling-dependent manner. Shortly after influenza virus infection, an increased number of PD-1 positive T cells are recruited to the airways. Inhibition of PD-1 signalling using monoclonal antibody blockade prevented CD8+cytotoxic T lymphocyte impairment, reduced viral titres during primary infection and enhanced protection of immunized mice against challenge infection (Erickson et al. 2012. J Clin Invest 122:2967-2982). Blockade of airway epithelial PD-L1 with antibodies improved CD8 T cell function, defined by increased production of IFN-γ and granzyme B, and expression of CD107ab. The PD-L1 blockade in the airways served to accelerate influenza virus clearance and enhance infection recovery (McNally et al. 2013, J Virol 87:12916-12924).

In December 2019, a novel coronavirus was first reported in Wuhan, China. It was named Severe Acute Respiratory Syndrome Coronavirus 2 (SARS-CoV-2) and is responsible for coronavirus disease 2019 (COVID-19). After the outbreak in China, the virus rapidly spread around the world with almost 400,000 reported infections and an approximate 14,000 deaths at the end of March 2020, with an anticipated significant increase. It was reported in a scientific publication that in patients with a SARS-CoV-2 infection the total number of Natural Killer cells and CD8+ T cells was markedly decreased, and that their function was exhausted with an increased expression of NKG2A. These results suggest that the functional exhaustion of cytotoxic lymphocytes was associated with the SARS-CoV-2 infection, and that the virus apparently breaks down the antiviral immunity at an early stage of infection (Zheng et al. 2020. Cell Mol Immunol 10.1038/s41423-020-0402-2). This effect was supported by earlier findings showing that in the acute phase of a SARS infection a severe reduction in the number of T cells in the blood was observed (Channappanavar et al. 2014. Immunol Res 58:118-128).

Despite the achievements with several monoclonal antibodies, the therapeutic effect of PD-1/PD-L1 antagonists is currently not satisfactory (Jiang et al. 2019, Molecular Cancer 18:10). In PD-L1 positive metastatic melanoma or lung cancer, the objective response rate of anti-PD-L1 antagonists is only 40-50%. In colorectal cancer, although the PD-L1 positive rate is 40-50%, anti-PD-1 or anti-PD-L1 drugs show very low efficacy (Sznol. 2014, Cancer J 20(4):290-295). Moreover, treatment with such immune checkpoint inhibitors is associated with a unique pattern of immune-related adverse effects or side effects. The effect of using monoclonal antibodies targeting the PD-1L/PD-L1 signalling process during (acute) viral respiratory infections remains to be elucidated. In conclusion, there is a desire for alternative methods and means to target the PD-1/PD-L1 interaction and pathway.

### SUMMARY OF THE INVENTION

Disclosed and claimed herein is an antisense oligonucleotide (AON) that is capable of inducing skipping at least exon 3 from CD274 pre-mRNA, wherein the AON comprises a sequence: that is substantially complementary to a sequence that is entirely within exon 3 of the CD274 gene; that is substantially complementary to a sequence of exon 3 of the CD274 gene and is substantially complementary to a sequence of the intron located upstream of exon 3, and thereby overlaps with the 5′ intron/exon boundary; or that is substantially complementary to a sequence of exon 3 of the CD274 gene and is substantially complementary to a sequence of the intron located downstream of exon 3, and thereby overlaps with the 3′ exon/intron boundary. Exon 3 of the human CD274 gene encodes the immunoglobulin variable (Igv)-like domain of PD-L1 that is critical for binding to PD-L1's natural receptor PD-1. In a preferred aspect of the invention, the AON of the invention comprises or consists of a sequence selected from the group consisting of: SEQ ID NO: 1, 2, 4, 7, 8, 9, 10, 11, and 12. In another preferred aspect, the AON comprises less than 26 nucleotides, preferably wherein the AON consists of 16, 17, 18, 19, 20, 21, 22, 23, 24, or 25 nucleotides. In yet another preferred aspect, the invention relates to an AON that is substantially complementary to a consecutive stretch of nucleotides within SEQ ID NO:20. In a particularly preferred embodiment, the AON of the invention comprises at least one non-naturally occurring chemical modification such as one or more modified internucleoside linkages and/or one or more modified sugar moiety. Particularly preferred modifications comprise phosphorothioate internucleoside linkages, 2′-O-methyl and/or 2′-methoxyethoxy modifications.

The present invention also relates to a pharmaceutical composition comprising an AON according to the invention, and a pharmaceutically acceptable carrier. In another embodiment, the invention relates to a viral vector, preferably an AAV vector, expressing an AON according to the invention. In one embodiment, the invention relates to an AON according to the invention, a pharmaceutical composition according to the invention, or a viral vector according to the invention, for use as a medicament, preferably in the treatment of an (auto-) immune disease, a cancer, a chronic or acute viral infection, more preferably a liver infection (such as those caused by HBV or HCV).

The invention also relates to an AON according to the invention for use in the treatment of a viral infection, preferably an acute viral infection, more preferably an acute respiratory viral infection caused by an influenza virus, a Severe Acute Respiratory Syndrome Coronavirus (SARS-CoV) or a Middle East Respiratory Syndrome coronavirus (MERS-CoV), or a derivative thereof. In an even more preferred aspect, the invention relates to an AON according to the invention for use in the treatment of an infection caused by SARS-CoV-2, or a derivative thereof.

The invention also relates to a method of inducing skipping of at least exon 3 from CD274 pre-mRNA in a cell, comprising the step of administering to the cell an AON according to the invention, a pharmaceutical composition according to the invention, or a viral vector according to the invention; optionally further comprising the step of determining whether the skip of exon 3 from the CD274 pre-mRNA has occurred. Preferably the cell is a human cell, and more preferably, the cell is an in vivo cell or a cell that is cultured in vitro or ex vivo. More preferably, the cell is a PD-L1 expressing cell, such as a T cell. In another aspect, the invention relates to a method of treating a viral infection, preferably an acute viral infection, more preferably an acute respiratory viral infection caused by an influenza virus, a Severe Acute Respiratory Syndrome Coronavirus (SARS-CoV) or a Middle East Respiratory Syndrome coronavirus (MERS-CoV), or a derivative thereof. In an even more preferred aspect, the invention relates to a method of treating an infection caused by SARS-CoV-2 or a derivative thereof. In one aspect, the method comprises the step of administering (preferably by direct administration, for instance using a nebulizer) to the airways of a subject in need thereof an AON according to the invention.

In one other embodiment, the invention relates to a method of modulating the function of PD-L1 in a cell, comprising the step of administering to the cell an AON according to the invention, a pharmaceutical composition according to the invention, or a viral vector according to the invention; and allowing the skip of at least exon 3 from the CD274 pre-mRNA that encodes the PD-L1 protein. In one other embodiment, the invention relates to the use of an AON according to the invention, a pharmaceutical composition according to the invention, or a viral vector according to the invention in the manufacture of a medicament for the treatment, prevention or amelioration of a viral infection, an auto-immune disease, or a cancer.

### BRIEF DESCRIPTION OF THE DRAWINGS

FIG. 1 shows (5′ to 3′) the sequence of exon 3 of the human CD274 gene (upper strand, bold) and the surrounding intron sequences (lower case), together with the 12 initially designed antisense oligonucleotides (AON1 to AON12, from 3′ to 5′, left to right) as disclosed herein. An AON of the prior art is also given (‘Guccione’). The sequences of AON1 to AON12 are SEQ ID NO:1, 2, 3, 4, 5, 6, 7, 8, 9, 10, 11, and 12, respectively. The full gene sequence as shown here (including intron and exon sequences) is SEQ ID NO:13. The bold exon 3 sequence is SEQ ID NO:14. The Guccione AON sequence is SEQ ID NO:15. The target sequence for AON9 and its derivatives/equivalents is underlined (SEQ ID NO:20). The DNA sequence of exon 3 and its surrounding sequences is given, but the skilled person understands that the corresponding pre-mRNA sequence is the target sequence for splice modulation by the AONs as disclosed herein.

FIG. 2 shows the results on a Bioanalyzer of PCR products generated on cDNA from RNA obtained from human hepatocellular carcinoma cells (HepG2) that were induced with IFN-γ and subsequently transfected with AON1 to AON12. The upper arrow shows the position of the 824 nt PCR product representing the wild type sequence full length (FL) without exon 3 skipping. The lower arrow shows the position of the 482 nt PCR product representing the mRNA from which exon 3 has been skipped (Δex3). Negative controls were a mock transfection (mock) using transfection reagents but no AON, no transfection (nt) and the use of a non-targeting control AON (Ctrl AON).

FIG. 3 shows the results on a Bioanalyzer of PCR products generated on cDNA from RNA obtained from human hepatocellular carcinoma cells (HepG2) that were induced with IFN-γ and subsequently transfected with AON1, AON7, AON9 and AON12, in duplicate. The upper arrow shows the position of the 824 nt PCR product representing the wild type sequence full length (FL) without exon 3 skipping. The lower arrow shows the position of the 482 nt PCR product representing the mRNA from which exon 3 has been skipped (Δex3). Negative controls were a mock transfection (mock) using transfection reagents but no AON, no transfection (nt) and the use of a non-targeting control AON (Ctrl AON), which was also performed in duplicate.

FIG. 4 shows the results on a Bioanalyzer of PCR products generated on cDNA from RNA obtained from HeLa cells that were induced with IFN-γ and subsequently transfected with AON1, AON7, AON9, AON12 and an AON known from the art (′Guccione′ described in WO 2019/004939, see FIG. 1), in duplicate. A non-targeting control AON was taken along as a negative control (Ctrl AON). All AONs were fully modified with 2′-OMe. The upper arrow shows the position of the 824 nt PCR product representing the wild type sequence full length (FL) without exon 3 skipping. The lower arrow shows the position of the 482 nt PCR product representing the mRNA from which exon 3 has been skipped (Δex3). Below the gel the percentages of skip are given based on intensities of the bands. These percentages (taking into account the partial skip of exon 4) were also averaged for the duplicates.

FIG. 5 shows the results on a Bioanalyzer of PCR products generated on cDNA from RNA obtained from HeLa cells that were induced with IFN-γ and subsequently transfected with AON1, AON7, AON9, AON12 and a non-targeting control AON as a negative control (Ctrl AON). A mock transfection (carrier only), and no transfection (NT) were also taken as negative controls. All AONs were fully modified with 2′-MOE (FIG. 4 shows the results with the 2′-OMe modified AONs). The upper arrow shows the position of the 824 nt PCR product representing the wild type sequence full length (FL) without exon 3 skipping. The lower arrow shows the position of the 482 nt PCR product representing the mRNA from which exon 3 has been skipped (Δex3). Below the gel the percentages of skip are given based on intensities of the bands. These percentages (considering the partial skip of exon 4) were also averaged for the duplicates.

FIG. 6 shows the results on a Bioanalyzer of PCR products generated on cDNA from RNA obtained from HeLa cells that were induced with IFN-γ and subsequently transfected with 2′-OMe modified AON9, AON9LNA, AON9.1, AON9.2, AON9.3, AON9.4, and with 2′-MOE modified AON9, AON9LNA, AON9.1, AON9.2, AON9.3, and AON9.4 (from left to right). The positions of the 824 nt PCR product and the 482 nt product from which exon 3 is skipped are as in FIG. 5. Below the gel the percentages of skip are given based on intensities of the bands. These percentages (considering the partial skip of exon 4) were also averaged for the duplicates.

FIG. 7 shows the results on a Bioanalyzer of PCR products generated on cDNA from RNA obtained from HeLa cells that were induced with IFN-γ and subsequently transfected with 2′-OMe modified AON12, AON12LNA, AON12.1, AON12.2, AON12.3, AON12.4, AON12.5, AON12.6 and with 2′-MOE modified AON12, AON12LNA, AON12.1, and AON12.2 (from left to right). The positions of the 824 nt PCR product and the 482 nt product from which exon 3 is skipped are as in FIG. 5. Below the gel the percentages of skip are given based on intensities of the bands. These percentages (considering the partial skip of exon 4) were also averaged for the duplicates.

FIG. 8 shows (A) proliferation of healthy donor derived T-cells after 48 hr (grey bars) and 72 hr (dark bars) co-culture with non-small cell lung cancer (NSCLC) cells that were either transfected with AON9.1 or a control (cntrl) oligonucleotide. Bars and error bars depict mean+SD of replicate fold change values versus control oligonucleotide. The value found with the control oligonucleotide was set as 1. The bars give an increase of proliferation, while fluorescence is in fact lowered, which means that the results are depicted reciprocally. Mean median fold increase (GMFI) of biological replicates (n=2) were statistically compared using 2-way ANOVA applying SIDAK's multiplicity correction. (B) shows the expression of PD-L1 on the transfected NSCLC cells determined with an anti-human CD274 antibody. Asterisks represent statistically significant change versus control. * p<0.05; ** p<0.01; **** p<0.0001.

### DETAILED DESCRIPTION OF THE INVENTION

As outlined above, monoclonal antibodies have been widely tested, and some have been commercially approved by regulatory bodies for modulation the function of PD-1 and PD-L1, predominantly for the treatment of certain cancers. Others have disclosed the use of short interfering nucleic acid (siNA), short interfering RNA (siRNA), double-stranded RNA (dsRNA), micro-RNA (miRNA) and short hairpin RNA (shRNA) molecules to modulate the expression of PD-L1 and PD-1 (WO2005/007855; WO2007/084865; U.S. Pat. No. 8,507,663; Dolina et al. 2013, Molecular Therapy-Nucleic Acids 2:e72). WO2006/042237 describes a method of diagnosing cancer by assessing PD-L1 expression in tumours and suggests delivering an agent, which interferes with the PD-1/PD-L1 interaction, to a patient. Such interfering agents were suggested to be antibodies, antibody fragments, siRNA or antisense oligonucleotides (AONs), but no specific examples were disclosed of such interfering agents. WO2017/157899 discloses the use of so-called “gapmers” for downregulating the expression of PD-L1 in liver cells. Gapmers are aimed at targeting an mRNA and thereby inducing nuclease breakdown of the double-stranded target/gapmer complex, as soon as the gapmer is bound to its target. WO2016/138278 discloses gene silencing compounds, such as two or more single stranded AONs that are linked at their 5′ ends, for the inhibition of immune checkpoints including PD-L1.

The inventors of the present invention decided to explore a different approach. The present invention relates to AONs that target human CD274 (pre-) mRNA for specifically skipping one or more exons from the CD274 (pre-) mRNA. The human CD274 gene encodes the human PD-L1 protein. The AONs of the present invention are not aimed at downregulation of protein expression, or at inducing nuclease breakdown of the target molecule, but rather at modulating the functionality of the protein translated from the mRNA from which the exon (or exons) is skipped. The ultimate aim is to prevent the ability of the resulting PD-L1 (in which the skipped exon part is absent) to interact with its natural receptor PD-1, thereby modulating (down-regulating) the effect of the PD-1/PD-L1 receptor/ligand pathway and thereby preventing T cell exhaustion. It is a specific aim of the present invention to downmodulate the function of PD-L1 in acute respiratory viral infections by providing AONs that can skip one or more exons (preferably exon 3) from the PD-L1 pre-mRNA in target airway epithelial cells. Downregulation of PD-L1 functioning (and therethrough downplaying its signalling through the interaction with PD-1 and thereby decreasing T cell exhaustion and/or apoptosis) results in an increased immune response towards viral infections in the airway and likely in more rapid viral clearing. It has been shown that T cell exhaustion occurs after infection of influenza viruses (Erickson et al. 2012; McNally et al. 2013) and after infections with SARS-CoV-2 resulting in COVID-19 (Zheng et al. 2020). To the best of the knowledge of the inventors of the present invention the use of AONs to skip an exon from PD-L1 pre-mRNA has not been disclosed or suggested for use in acute respiratory viral infections such as COVID-19. The AONs of the present invention are therefore useful in the treatment of (acute and chronic) viral infections, as well as in cancers in which T cell exhaustion prevents the removal of—by the patient's own immune system—virus-infected cells and cancer cells. In a preferred aspect, the AONs of the present invention induce the skipping of exon 3 from the CD274 pre-mRNA. Exon skipping is often used as a means to restore the function of proteins, where mutations cause for instance the inclusion of an aberrant exon in the mRNA (e.g. WO2016/135334 for skipping an aberrant 128 bp exon from the human CEP290 gene; and WO2017/186739 for skipping a pseudo exon from the human USH2A gene). Exon skipping is also useful in skipping in-frame exons that harbour mutations causing a disease (e.g. WO2018/055134 for skipping mutated exon 13 from human USH2A pre-mRNA), thereby restoring the functionality of the protein. The present invention is directed at AONs that cause skipping of one or more exons from a pre-mRNA to alter the functionality of a protein, thereby downplaying its normal function by skipping (in the case of exon 3) an in-frame part from the human CD274 pre-mRNA. Skipping exon 3 renders the resulting PD-L1 protein unable to interact with its natural receptor PD-1. However, skipping exon 3 from the CD274 pre-mRNA does not necessarily mean that the expression of the protein is influenced, negatively or positively. It may be that the protein is expressed to similar levels as the wild type version. The inventors envision at least that, since the immunoglobulin (Igv)-like domain is absent, that the interaction with PD-1 is not taking place. PD-L1 lacking the Igv-like domain may still function in other processes, which may need to be addressed further. It is noted that AONs for modulating the function of a T cell have been disclosed in the art. WO2019/004939 describes AONs that target an extensive variety of AONs targeting IFN-γ, granzyme, perforin 1, PRDM1, CD40LG, NDFIP1, PDCD1 LG2, REL, BTLA, CD80, CD160, CD244, LAG3, TGIT, ADORA2A, TIM-3, as well as PD-1 and PD-L1 RNAs, for—in some cases—skipping exons. More than 70,000 oligonucleotides are disclosed therein, including approximately 1760 AONs that target exon 3 of CD74, none of which were shown to work. Table 1 and 2 in WO2019/004939 show a single AON (SEQ ID NO:19411 therein) that supposedly can be used for exon 3 skipping of human CD274 pre-mRNA and an AON (SEQ ID NO:20993 therein) that supposedly can be used for exon 4 skipping of human CD274 pre-mRNA. But, no reasoning was given for taking these two AONs from the laundry list of oligonucleotide sequences and mention them separately and no experimental data was presented that showed that exon skipping was in fact achieved for PD-L1, although—on the contrary—exon skipping was demonstrated for IFN-γ, granzyme B (GZMB), perforin (PRF), PD-1, CD244, TM-3, TGIT, PRDM1, REL, CD160, and CD80 RNAs. The inventors of the present invention have sought for alternative PD-L1 targeting AONs that are capable of exon 3 skipping. The inventors here show that not all AONs are able to prevent the inclusion of exon 3 in the CD274 mRNA. But, in contrast to what was shown in the prior art, the inventors were able to find certain specific areas within exon 3 of human CD274 and areas including the intron/exon boundaries that could be targeted to obtain proper exon 3 skipping. The inventors of the present invention have also taken the AON of the prior art that could supposedly be used for exon 3 skipping along (herein referred to as the ‘Guccione’ AON; SEQ ID NO:19411 of WO2019/004939) and compared it to the AONs that were newly designed and herein. Notably, the Guccione AON almost completely failed in giving exon 3 skipping from human CD274 pre-mRNA, while in contrast several of the AONs identified and generated by the inventors of the present invention were capable of giving very significant skip, even in some instances up to almost 90% efficiency (based on Bioanalyzer results; see Examples section herein). Hence, it is concluded that this is the first time that actual exon 3 skipping from human CD274 pre-mRNA was achieved and that the inventors of the present invention were the first to identify AONs that could give exon 3 skipping.

The inventors of the present invention reasoned that skipping exon 3 from human CD274 pre-mRNA would result in a shorter PD-L1, because exon 3 has a length of 342 nucleotides and is in-frame with exon 2 and 4. Human PD-L1 is translated from 7 exons present in the human CD274 gene. Exon 3 encodes the extracellular Igv-like domain which is critical for binding to PD-1 and any isoforms lacking completely or partially this domain do not bind to PD-1 (Carreno and Collins. 2002, Annu Rev Immunol 20: 29-53). Therefore, exclusion of exon 3 alone is expected to inhibit PD1/PDL1 signalling. In addition, He and Liu (2005, Acta Pharmacologica Sinica 26(4):462-468) found that PD-L1delEx3 is retained in the cytoplasm/ER probably caused by mis-folding of the protein and/or an altered post-translational glycosylation pattern. When not on the cell surface PD-L1 is not able to interact with PD-1 and cause T-cell exhaustion. It is noted that He and Liu (2005) identified 6 exons: GenBank AL162253 (similar to exon 2-7 as used in the present invention), likely because exon 1 is in the 5′ UTR, and does not translate into protein, which means that the PD-L1delEx3 referred to above is in fact a transcript lacking exon 2 in He and Liu (2005).

The present invention relates to an antisense oligonucleotide (AON) for modulating the function of a T cell. More in particular, the AON modulates the function of a T cell by modulating the ability of PD-L1 in a target cell to interact with its receptor PD-1 presented on the surface of a T cell. The AON of the present invention modulates, and preferably negatively influences the ability of PD-L1 to interact with PD-1 by causing a skip of at least exon 3 from the pre-mRNA that encodes PD-L1. The absence of the protein part (immunoglobulin (Igv)-like domain) that is encoded by exon 3 causes the resulting PD-L1 polypeptide non-functional in its interaction with PD-1 and through this, the AON modulates the function of PD-L1 and thereby influences the negative effects (such as exhaustion and apoptosis) of the T cell that interacts with its PD-1 receptor to the target cell. Preferably the AON of the present invention thereby inhibits, diminishes and/or prevents T cell exhaustion, preferably during (auto-) immune disease, (acute and/or chronic) viral infections or in the occurrence of cancer. In a more preferred aspect, the AON of the present invention inhibits, diminishes and/or prevents T cell exhaustion during an acute respiratory viral infection caused by an influenza virus or a coronavirus. The AON of the present invention is capable of inducing skipping of at least exon 3 from CD274 pre-mRNA, preferably human CD274 pre-mRNA, wherein the AON comprises a sequence that is substantially complementary to a sequence that is entirely within exon 3 of the CD274 gene, or wherein the AON comprises a sequence that is substantially complementary to a sequence of exon 3 of the CD274 gene and that is substantially complementary to a sequence of the intron located at the 5′ or the 3′ side of exon 3, and thereby overlaps with the 5′ or 3′ intron/exon boundary, respectively. The complementary sequence is preferably consecutive with a full consecutive match for all nucleotides in the complementary AON. It should be noted that the skilled person, based on the present teaching is able to determine what ‘capable of inducing skipping at least exon 3 from CD274 pre-mRNA’ means. Such can be determined by using RT-PCR on RNA obtained from cells into which the AON is introduced (either by transfection, gymnotic uptake, or otherwise) in which the PCR reveals whether exon 3 is absent or present. One non-limiting example of such an assessment, as outlined herein, shows that some AONs are incapable of causing exon 3 skip (revealing 0% skip according to the Bioanalyzer results), while other AONs cause a skip efficiency reaching almost 90%. The invention relates to an AON that can block an immune checkpoint molecule from performing its normal function. In one embodiment, the AON of the present invention is complementary to a target sequence that is entirely within exon 3 of CD274 pre-mRNA, preferably human CD274 pre-mRNA. In another embodiment, the AON of the present invention is complementary to a continuous target sequence that is partly within exon 3 of the CD274 pre-mRNA and partly within the upstream intron of exon 3, and therefore also targets the intron/exon boundary at the 5′ end of exon 3 of CD274. In yet another embodiment, the AON of the present invention is complementary to a continuous target sequence that is partly within exon 3 of the CD274 pre-mRNA and partly within the downstream intron of exon 3, and therefore also targets the exon/intron boundary at the 3′ end of exon 3 of CD274. The length of complementarity differs from AON to AON but can be determined by the skilled person based on the current teaching. Preferably, the complementarity is 100%, but may be less if the AON is capable of inducing exon 3 skip from human CD274 pre-mRNA. In a preferred embodiment the AON of the present invention comprises or consists of a sequence selected from the group consisting of: SEQ ID NO: 1, 2, 4, 7, 8, 9, 10, 11, and 12. More preferably, the AON of the present invention comprises or consists of a sequence selected from the group consisting of: SEQ ID NO: 1, 7, 9, and 12. In a particularly preferred embodiment, the AON of the present invention comprises less than 26 nucleotides, and preferably consists of 16, 17, 18, 19, 20, 21, 22, 23, 24, or 25 nucleotides. In one particularly preferred embodiment, the AON of the present invention relates to a 16, 17, 18, 19, 20, 21, 22, 23, 24, or 25-nucleotide long AON that is 100% complementary to a consecutive stretch of nucleotides within the sequence of SEQ ID NO:20. The single AON known from the art, and which was designed to give exon 3 skipping (see WO 2019/004939; and the accompanying examples herein) comprises 26 nucleotides and did not show exon 3 skipping after administration to cells that were induced with IFN-γ, in contrast to a number of the AONs of the present invention, that showed high exon 3 skipping efficiencies.

In one embodiment, the present invention relates to AONs that are derivatives of the AONs of the present invention (for instance those that comprise additional nucleotides on either end, or that are made shorter by removal of nucleotides on either end), as long as their functionality (inducing the skip of at least exon 3 from CD274 pre-mRNA) remains present and can be determined and reaches a significant level above background (for instance above 0% as calculated on the Bioanalyzer results, as outlined in the accompanying examples, which showed that an AON from the art was not able to give exon 3 skipping above 0%).

In another preferred embodiment, the AON of the present invention comprises at least one non-naturally occurring chemical modification. Preferably, the non-naturally occurring modification comprises a modification of at least one internucleoside linkage. Preferred internucleoside linkage modifications are non-bridging oxygen atom substituting a sulfur atom, a phosphonate, a phosphorothioate, a phosphodiester, a phosphoromorpholidate, a phosphoropiperazidate, a phosphonoacetate, a methylphosphonate, and a phosphoroamidate. The linkage modification comprises more preferably a phosphorothioate. In an even more preferred embodiment, all internucleoside linkages are chemically modified by a non-naturally occurring modification, and in a most preferred embodiment, all internucleoside linkages within the AON of the present invention carry a phosphorothioate modification. The Sp or Rp configuration of each of these phosphorothioate linkage modifications may be carefully selected to increase the binding efficiency to its target sequence, as well as its stability in vivo.

In another preferred embodiment, the AON of the present invention comprises one or more sugar moieties that is mono- or di-substituted at the 2′, 3′ and/or 5′ position, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy. Preferably, the AON comprises at least one sugar moiety carrying a 2′-OMe modification. In another preferred aspect the AON comprises at least one sugar moiety carrying a 2′-MOE modification. 2′-OMe and 2′-MOE modifications may both be present in a single AON of the present invention. In another aspect, the AON is fully modified with 2′-OMe or fully modified with 2′-MOE. The activity of each type of modified AON can be easily determined by the skilled person based on the teaching provided herein. As can be shown in the examples below, while one particular AON carrying all 2′-OMe modifications is more efficient in exon 3 skipping than its 2′-MOE counterpart, such may also be the other way around, wherein the full 2′-MOE modified version works more efficient that its 2′-OMe counterpart. The skilled person knows that such may depend on the cell type that is used, the way of introducing an AON into a cell, cell cycle state, etc. and that for each setting such may be tested and adjusted, which is all within the capabilities of the person skilled in the art.

In another embodiment, the invention relates to an AON according to the invention, wherein the AON is chemically linked to one or more conjugates that enhance the activity, the cellular distribution, or cellular uptake of the AON. Particularly preferred conjugates that may be linked to the AON of the present invention are carbohydrates to enhance the delivery to liver cells. Such may be particularly useful in the treatment of diseases that influence the function of liver cells, such as with (chronic) liver infections by viruses such as HBV and HCV. WO 93/07883 and WO2013/033230 provide suitable conjugates, which are hereby incorporated by reference. Further suitable conjugate moieties are those capable of binding to the asialoglycoprotein receptor, in particular tri-valent N-acetylgalactosamine conjugate moieties are suitable for binding to this receptor (see e.g. WO2014/076196, WO2014/207232 and WO 2014/179620, hereby incorporated by reference). In one embodiment, the conjugate is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

In another embodiment, the invention relates to a pharmaceutical composition comprising an AON according to the invention, and a pharmaceutically acceptable carrier. In yet another embodiment, the invention relates to a viral vector expressing an AON according to the invention. Preferred viral vectors that can deliver AONs, encoded by the nucleic acid that they carry, are adeno-associated viruses (AAVs). In one aspect, the invention relates to an AON according to the invention, a pharmaceutical composition according to the invention, or a viral vector according to the invention, for use as a medicament.

The invention, in yet another embodiment, relates to an AON according to the invention for use in the treatment of a viral infection, an auto-immune disease or cancer. Preferably, the AON of the invention is for use in the treatment of a viral infection, preferably a liver infection, such as those caused by HBV and HCV. Also, in a preferred aspect, the AON of the invention is for use in the treatment, prevention, or amelioration of a cancer, for instance those caused by viruses, such as HBV-induced HCC and EBV-induced Non-Hodgkin Lymphomas. In yet another preferred aspect, the AON of the present invention is for use in the treatment, prevention, or amelioration of a non-virally caused cancer, such as non-small lung cancer, head and neck squamous cell carcinoma, squamous cell lung cancer, renal carcinoma, Hodgkin's lymphoma, cutaneous squamous cell carcinoma, urothelial carcinoma, metastatic merkel-cell carcinoma, and unresectable non-small cell lung cancer, which are non-limiting examples in which T cell exhaustion plays a role, and in which the modulation of PD-L1 function may have a beneficial impact, after administering the AON of the present invention.

The invention also relates to an AON as outlined herein for use in the treatment of an acute viral infection, more preferably an acute respiratory viral infection caused by an influenza virus, a Severe Acute Respiratory Syndrome Coronavirus (SARS-CoV) or a Middle East Respiratory Syndrome coronavirus (MERS-CoV), or a derivative thereof. In an even more preferred aspect, the invention relates to an AON according to the invention for use in the treatment of an infection caused by SARS-CoV-2, or a derivative thereof. A derivative is defined as a viral strain that has become mutated over time (for instance while spreading throughout the human population, or in other mammals), and that may be infectious and capable of causing disease in mammals that did or did not experience an earlier infection with SARS-CoV-1, SARS-CoV-2 or MERS-CoV. As an example, if the SARS-CoV-2 virus is mutated such that it may not be recognized (and/or neutralized) by natural or recombinant antibodies that were raised against the SARS-CoV-2 virus in an earlier epidemic/pandemic, such a mutated (new) virus is considered a derivative of SARS-CoV-2. In another aspect, the invention relates to a method of treating a viral infection, preferably an acute viral infection, more preferably an acute respiratory viral infection caused by an influenza virus, a Severe Acute Respiratory Syndrome Coronavirus (SARS-CoV) or a Middle East Respiratory Syndrome coronavirus (MERS-CoV), or a derivative thereof. In an even more preferred aspect, the invention relates to a method of treating an infection caused by SARS-CoV-2 or a derivative thereof. In one aspect, the method comprises the step of administering (preferably by direct administration, for instance using a nebulizer) to the airways of a subject in need thereof an AON according to the invention.

The AON of the present invention is not a gapmer, which in general comprises both RNA and DNA. The AON of the present invention can induce the skip of at least exon 3 from human CD274 pre-mRNA; it is not aimed at downregulation PD-L1 expression. However, it cannot be excluded that skipping exon 3 from the CD274 pre-mRNA also influences protein expression. The AON of the present invention is not limited by the functional feature of influencing protein expression, although it should be capable of inducing the skip of at least exon 3 from the human CD274 pre-mRNA (as discussed above), and as can be determined by the methods described herein and by using the general common knowledge and methodologies known to the person skilled in the art.

In a preferred embodiment, the AON of the present invention is delivered ‘as is’, or ‘naked’. Nevertheless, the art contains multiple ways of delivering AONs to cells, either in vitro, ex vivo or in vivo. Depending on the disease, disorder or infection that needs to be treated, or on the cell, tissue or part of the body that needs to be reached by the AON of the present invention, an administration route or delivery method may be selected. Examples for delivery when the AON is not delivered naked, are delivery agents (including viral vectors encoding the AON) or delivery vehicles such as nanoparticles, like polymeric nanoparticles, liposomes, antibody-conjugated liposomes, cationic lipids, polymers, or cell-penetrating peptides. For delivery in the airways such as the lung, the AON of the present invention may be delivered in a suitable delivery vehicle for efficient targeting of airway epithelial cells.

In another embodiment, the invention relates to a method of inducing skipping of at least exon 3 from CD274 pre-mRNA in a cell, comprising the step of administering to the cell an AON according to the invention, a pharmaceutical composition according to the invention, or a viral vector according to the invention; optionally further comprising the step of determining whether the skip of exon 3 from the CD274 pre-mRNA has occurred. Determining whether skipping of exon 3 has occurred can be performed by different means, such as sequencing the PCR product obtained from RNA from the treated cell, or by RT-PCR and determining the size of the PCR product (as outlined herein). One can also determine the skip using a functional assay, for instance by determining whether a T cell still suffers from exhaustion, or whether the resulting PD-L1 protein, encoded by the pre-mRNA is (in)-capable of interacting with its natural receptor, PD-1 at the T cell that is otherwise exhausted or going towards apoptosis. In a preferred embodiment, the cell used in the method of the present invention is an in vivo cell, or when cultured, an in vitro or ex vivo human cell, more preferably a target in vivo cell that is infected by a virus, such as an epithelial cell in the case of a respiratory virus. In yet another embodiment, the invention relates to a target cell transformed or transfected with an AON according to the invention.

In another embodiment, the invention relates to a method of treating a human subject suffering from a disease related to T cell exhaustion, comprising the step of administering to the human subject an AON according to the invention, a pharmaceutical composition according to the invention or a viral vector according to the invention. Preferred diseases that are related to T cell exhaustion are (acute or chronic) viral infections, such as respiratory infections caused by an influenza or a coronavirus, liver infections caused by HBV or HCV, (auto-) immune disorders and cancers, such as virally induced cancers or cancers such as (unresectable) non-small lung cancer, head and neck squamous cell carcinoma, squamous cell lung cancer, renal carcinoma, Hodgkin's lymphoma, urothelial carcinoma and cutaneous squamous cell carcinoma.

In one embodiment, the invention relates to a method of modulating the function of PD-L1 in a target cell, comprising the step of administering to the cell an AON according to the invention, a pharmaceutical composition according to the invention, or a viral vector according to the invention; and allowing the skip of at least exon 3 from the CD274 pre-mRNA that encodes the PD-L1 protein. Preferably, the cell is a human cell. More preferably, the method is for modulating the function of PD-L1 by removal of the protein part encoded by exon 3, through exon skipping, thereby disabling the resulting PD-L1 protein product to interact with PD-1, and thereby preventing, or inhibiting, or ameliorating T cell exhaustion. Hence, the present invention relates to a method of treating, preventing, or ameliorating a disease, or disorder that is related to T cell exhaustion. As outlined herein, a wide variety of diseases and disorders exist in which T cell exhaustion plays a central role. It is the purpose of the invention to provide AONs that are useful in the treatment, prevention or amelioration of all such diseases, preferably (acute or chronic) viral infections, especially those of the respiratory tract, liver, (auto-) immune diseases, and cancers.

In yet another embodiment, the invention relates to a the use of an AON according to the invention, a pharmaceutical composition according to the invention, or a viral vector according to the invention in the manufacture of a medicament for the treatment, prevention or amelioration of an acute or chronic viral infection, an auto-immune disease, or a cancer. The preferred chronic or acute viral infections, (auto-) immune diseases and cancers that are preferably treated by said use are as outlined herein.

In a preferred aspect the AON of the present invention is an oligoribonucleotide. In a further preferred aspect, the AON according to the invention comprises at least one 2′-O alkyl modification, preferably a 2′-O-methyl (2′-OMe) modified sugar. In a more preferred embodiment, all nucleotides in said AON are 2′-OMe modified. In another preferred aspect, the invention relates to an AON comprising at least one 2′-O-methoxyethyl (2′-methoxyethoxy or 2′-MOE) modification. In a more preferred embodiment, all nucleotides of said AON carry a 2′-MOE modification. In yet another aspect the invention relates to an AON, wherein the AON comprises at least one 2′-OMe and at least one 2′-MOE modification. More preferably, the positions of the 2′-OMe and 2′-MOE modifications, when both present in the AON of the present invention in different nucleotides within the AON, are selectively chosen to achieve the highest skipping efficiency.

In another preferred embodiment, the AON according to the present invention has at least one non-naturally occurring modification, preferably a non-naturally occurring internucleoside linkage modification. A preferred non-naturally occurring internucleoside modification is a modification with phosphorothioate (a phosphorothioate linkage), a phosphonoacetate or a methylphosphonate. In a more preferred aspect, all sequential nucleotides of the AON of the present invention are interconnected by phosphorothioate linkages.

In yet another aspect, the invention relates to a pharmaceutical composition comprising an AON according to the invention, and a pharmaceutically acceptable carrier.

In another embodiment, the invention relates to a viral vector expressing an AON according to the invention. In another embodiment, the invention relates to an AON according to the invention, a pharmaceutical composition according to the invention, or a viral vector according to the invention, for use as a medicament, preferably for the use in immune therapy, more preferably to prevent, inhibit, ameliorate or treat a disease related to T cell exhaustion, such as (acute or chronic) viral infections, or cancer. In another embodiment, the invention relates to an AON according to the invention, a pharmaceutical composition according to the invention, or a viral vector according to the invention, for preventing, inhibiting, ameliorating or treating a disease related to T cell exhaustion, such as (acute or chronic) viral infections, (auto-) immune diseases, or cancers. Especially preferred are disease in which the PD-1/PD-L1 pathway is active and causes T cell exhaustion, such as seen in respiratory tract infections by influenza viruses and coronaviruses. The AONs of the present invention are useful in downregulating the PD-1/PD-L1 pathway by targeting the CD274 pre-mRNA (encoding human PD-L1) and thereby modulating the intracellular trafficking of the PD-L1 protein to the cell membrane and/or its function in interacting with its receptor PD-1 present on a T cell.

The invention also relates to a use of an AON according to the invention, a pharmaceutical composition according to the invention, or a viral vector according to the invention for the preparation of a medicament. Preferably, said medicament is for preventing, inhibiting, ameliorating or treating a disease related to T cell exhaustion, such as (auto-) immune disease, (acute or chronic) viral infections, or cancer. Preferred (acute or chronic) viral infections that may be treated with the AONs of the present invention are influenza virus, coronavirus (such as SARS-CoV-1, SARS-CoV-2 and MERS-CoV), HBV, HCV, HIV, HDV, parasite (e.g. malaria, toxoplasmosis) and LCMV infections. Preferred cancers that may be treated with the AONs of the present invention are cancers that escape the immune system of the patient by exhausting the T cells, and in which PD-1 and/or PD-L1 expression is upregulated, and wherein the increased activity of the PD-1/PD-L1 pathway results in such T cell exhaustion. Non-limiting examples of such cancers are (unresectable) non-small lung cancer, head and neck squamous cell carcinoma, squamous cell lung cancer, renal carcinoma, Hodgkin's lymphoma, urothelial carcinoma and cutaneous squamous cell carcinoma. The skilled person is aware of cancers in which T cell exhaustion is the, or one of the causes through which the tumour escapes the patient's immune system. Any of such tumour types may be targeted with one or more of the AONs of the present invention, to alleviate the effect of T cell exhaustion that occurs during the maintenance and/or growth of such tumours.

### Definitions

In all embodiments of the invention, the terms ‘modulating splicing’ and ‘exon skipping’ are synonymous. In respect of CD274, ‘modulating splicing’ or ‘exon skipping’ are herein to be construed as the exclusion of at least exon 3 from the human CD274 mRNA.

The term ‘exon skipping’ is herein defined as inducing, producing or increasing production within a cell of a mature mRNA that does not contain a particular exon (in the current case exon 3 of the CD274 gene) that would be present in the mature mRNA without exon skipping. Exon skipping is achieved by providing a cell expressing the pre-mRNA of said mature mRNA with a molecule capable of interfering with sequences such as, the (cryptic) splice donor or (cryptic) splice acceptor sequence required for allowing the enzymatic process of splicing, or with a molecule that is capable of interfering with an exon inclusion signal required for recognition of a stretch of nucleotides as an exon to be included in the mature mRNA; such molecules are herein referred to as ‘exon skipping molecules’, as ‘exon 3 skipping molecules’, as ‘exon skipping AONs’, or as ‘AONs capable of skipping exon 3 from human CD274 pre-mRNA’, or as ‘AONs capable of reducing the inclusion of exon 3 in human CD274 mRNA’. The term ‘pre-mRNA’ refers to a non-processed or partly processed precursor mRNA that is synthesized from a DNA template of a cell by transcription, such as in the nucleus. The term ‘mRNA’ refers to a processed RNA molecule that is translated to a protein in the cytoplasm of the cell, preferably, according to the present invention, lacking exon 3 when it concerns a CD274 mRNA.

The term ‘antisense oligonucleotide’ (AON) is understood to refer to a nucleotide sequence which is substantially complementary to, and hybridizes to, a (target) nucleotide sequence in a gene, a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable double stranded hybrid with the target nucleotide sequence in the (pre-) mRNA molecule under physiological conditions. The terms ‘AON’, ‘antisense oligonucleotide’, ‘oligonucleotide’ and ‘oligo’ are used interchangeably herein and are understood to refer to an oligonucleotide comprising an antisense sequence in respect of the target sequence. The AON of the present invention are not double stranded and are therefore not siRNAs. The AON of the present invention is man-made, and is chemically synthesized, generally in a laboratory by solid-phase chemical synthesis, followed by purification. It is typically purified or isolated.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g. about 10 μg) preferably means that the value may be the given value (of 10 μg) more or less 0.1% of the value.

In all embodiments of the present invention, the term “treatment” is understood to include the prevention, amelioration, cure and/or delay of the disease or condition.

The term “complementary” as used herein includes “fully complementary” and “substantially complementary”, meaning there will usually be a degree of complementarity between the oligonucleotide and its corresponding target sequence of more than 80%, preferably more than 85%, still more preferably more than 90%, most preferably more than 95%. For example, for an oligonucleotide of 20 nucleotides in length with one mismatch between its sequence and its target sequence, the degree of complementarity is 95%. Many naturally occurring variants are known in the PD-L1 gene (see for a list WO2017/157899, incorporated by reference herein), which means that when such a naturally occurring variant is targeted, the AON of the present invention may not be full complementary to that variant, but still be active in inducing exon skipping. In another form, the sequence of the AON may be adjusted to become 100% complementary to the naturally occurring variant. The term ‘substantially complementary’ used in the context of the invention indicates that some mismatches in the antisense sequence are allowed if the functionality, i.e. inducing skipping of at least exon 3 of the CD274 pre-mRNA is still acceptable. Preferably, the complementarity is from 90% to 100%. In general, this allows for 1 or 2 mismatches in an AON of 20 nucleotides or 1, 2, 3 or 4 mismatches in an AON of 40 nucleotides, or 1, 2, 3, 4, 5, or 6 mismatches in an AON of 60 nucleotides, etc. The degree of complementarity of the antisense sequence is preferably such that a molecule comprising the antisense sequence can anneal to the target nucleotide sequence in the RNA molecule under physiological conditions, thereby facilitating exon skipping. It is well known to a person having ordinary skill in the art, that certain mismatches are more permissible than others, because certain mismatches have less effect on the strength of binding, as expressed in terms of melting temperature or Tm, between AON and target sequence, than others. Certain non-complementary base pairs may form so-called “wobbles” that disrupt the overall binding to a lesser extent than true mismatches. The length of the AON also plays a role in the strength of binding; longer AONs having higher melting temperatures as a rule than shorter AONs, and the G/C content of an oligonucleotide is also a factor that determines the strength of binding, the higher the G/C content the higher the melting temperature for any given length. Certain chemical modifications of the nucleobases or the sugar-phosphate backbone, as contemplated by the present invention, may also influence the strength of binding, such that the degree of complementarity is only one factor to be taken into account when designing an oligonucleotide according to the invention.

The term “modulation of functionality” as referred to herein is to be understood as an overall term for an AON's ability to alter the function of PD-L1, or its natural processing (such as intracellular trafficking) in the cell, or its ability to interact with PD-1. Modulation of functionality may be determined by reference to a control experiment, for instance by using a non-targeting, non-related control AON, or mock transfection. Preferably, modulation of functionality by any of the AONs of the present invention renders PD-L1 less capable of giving T cell exhaustion through the PD-1/PD-L1 pathway. It may be that exon skipping within the PD-L1 pre-mRNA results in a decrease of expression of the (shortened) PD-L1 protein, or increased breakdown of the resulting mRNA or (shortened) protein.

The terms “adenine”, “guanine”, “cytosine”, “thymine”, “uracil” and hypoxanthine (the nucleobase in inosine) refer to the nucleobases as such. The terms adenosine, guanosine, cytidine, thymidine, uridine and inosine, refer to the nucleobases linked to the (deoxy)ribosyl sugar. The term “nucleoside” refers to the nucleobase linked to the (deoxy)ribosyl sugar.

### Modifications

The skilled person knows that an oligonucleotide, such as an RNA oligonucleotide, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine or uracil, or a derivative thereof. Cytosine, thymine and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate, such a moiety is still referred to as the backbone linkage of the monomer. This is referred to as a “backbone linkage modification”. In general terms, the backbone of an oligonucleotide comprises alternating scaffolds and backbone linkages.

In one aspect, the nucleobase in an AON of the present invention is adenine, cytosine, guanine, thymine, or uracil. In another aspect, the nucleobase is a modified form of adenine, cytosine, guanine, or uracil. In another aspect, the modified nucleobase is hypoxanthine (the nucleobase in inosine), pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and Inosine′ refer to the nucleobases linked to the (deoxy)ribosyl sugar. The term ‘nucleoside’ refers to the nucleobase linked to the (deoxy)ribosyl sugar. The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, and the like. The sugar moiety can be a pyranose or derivative thereof, or a deoxypyranose or derivative thereof, preferably ribose or derivative thereof, or deoxyribose or derivative thereof. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-O, 4′-C-ethylene-bridged nucleic acid (Morita et al. 2001. Nucleic Acid Res Supplement No. 1:241-242).

Sometimes the terms adenosine and adenine, guanosine and guanine, cytosine and cytidine, uracil and uridine, thymine and thymidine, inosine and hypoxanthine, are used interchangeably to refer to the corresponding nucleobase, nucleoside or nucleotide. Sometimes the terms nucleobase, nucleoside and nucleotide are used interchangeably, unless the context clearly requires differently. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

In one aspect, an AON of the present invention comprises a 2′-substituted phosphorothioate monomer, preferably a 2′-substituted phosphorothioate RNA monomer, a 2′-substituted phosphate RNA monomer, or comprises 2′-substituted mixed phosphate/phosphorothioate monomers. It is noted that DNA is considered as an RNA derivative in respect of 2′ substitution. An AON of the present invention comprises at least one 2′-substituted RNA monomer connected through or linked by a phosphorothioate or phosphate backbone linkage, or a mixture thereof. The 2′-substituted RNA preferably is 2′-F, 2′-H (DNA), 2′-O-Methyl or 2′-O-(2-methoxyethyl). The 2′-O-Methyl is often abbreviated to “2′-OMe” and the 2′-O-(2-methoxyethyl) moiety is often abbreviated to “2′-MOE”. In a preferred embodiment of this aspect is provided an AON according to the invention, wherein the 2′-substituted monomer can be a 2′-substituted RNA monomer, such as a 2′-F monomer, a 2′-NH₂ monomer, a 2′-H monomer (DNA), a 2′-O-substituted monomer, a 2′-OMe monomer or a 2′-MOE monomer or mixtures thereof. Preferably, any other 2′-substituted monomer within the AON is a 2′-substituted RNA monomer, such as a 2′-OMe RNA monomer or a 2′-MOE RNA monomer, which may also appear within the AON in combination.

Throughout the application, a 2′-OMe monomer within an AON of the present invention may be replaced by a 2′-OMe phosphorothioate RNA, a 2′-OMe phosphate RNA or a 2′-OMe phosphate/phosphorothioate RNA. Throughout the application, a 2′-MOE monomer may be replaced by a 2′-MOE phosphorothioate RNA, a 2′-MOE phosphate RNA or a 2′-MOE phosphate/phosphorothioate RNA. Throughout the application, an oligonucleotide consisting of 2′-OMe RNA monomers linked by or connected through phosphorothioate, phosphate or mixed phosphate/phosphorothioate backbone linkages may be replaced by an oligonucleotide consisting of 2′-OMe phosphorothioate RNA, 2′-OMe phosphate RNA or 2′-OMe phosphate/phosphorothioate RNA. Throughout the application, an oligonucleotide consisting of 2′-MOE RNA monomers linked by or connected through phosphorothioate, phosphate or mixed phosphate/phosphorothioate backbone linkages may be replaced by an oligonucleotide consisting of 2′-MOE phosphorothioate RNA, 2′-MOE phosphate RNA or 2′-MOE phosphate/phosphorothioate RNA.

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e. the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-O-methyl, 2′-O-(2-cyanoethyl), 2′-MOE, 2′-0-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-0-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-0,4′-C) constrained ethyl (cEt) BNA monomer, a (2′-O,4′-C) constrained methoxyethyl (cMOE) BNA monomer, a 2′,4′-BNA^{NC} (NH) monomer, a 2′,4′-BNA^{NC} (NMe) monomer, a 2′,4′-BNA^{NC} (NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above.

A “backbone modification” indicates the presence of a modified version of the ribosyl moiety (“scaffold modification”), as indicated above, and/or the presence of a modified version of the phosphodiester as naturally occurring in RNA (“backbone linkage modification”). Examples of internucleoside linkage modifications are phosphorothioate (PS), chirally pure phosphorothioate, Rp phosphorothioate, Sp phosphorothioate, phosphorodithioate (PS2), phosphonoacetate (PACE), thophosphonoacetate, phosphonacetamide (PACA), thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphoryl guanidine (PGO), methylsulfonyl phosphoroamidate, phosphoramidite, phosphonamidite, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino (MMI), and thioacetamido (TANA); and their derivatives.

The present invention also relates to a chirally enriched population of modified AONs according to the invention, wherein the population is enriched for modified AONs comprising at least one particular phosphorothioate internucleoside linkage having a particular stereochemical configuration, preferably wherein the population is enriched for modified AONs comprising at least one particular phosphorothioate internucleoside linkage having the Sp configuration, or wherein the population is enriched for modified AONs comprising at least one particular phosphorothioate internucleoside linkage having the Rp configuration.

In a preferred embodiment, the nucleotide analogue or equivalent comprises a modified backbone, exemplified by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells.

It is further preferred that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA molecule contains no charged phosphate groups, PNA-RNA hybrids are usually more stable than RNA-RNA or RNA-DNA hybrids, respectively (Egholm et al. (1993) Nature 365:566-568).

It is understood by a skilled person that it is not necessary for all positions in an AON to be modified uniformly. In addition, more than one of the analogues or equivalents may be incorporated in a single AON or even at a single position within an AON. In certain embodiments, an AON of the invention has at least two different types of analogues or equivalents. A preferred exon skipping AON according to the invention comprises a 2′-O alkyl phosphorothioated antisense oligonucleotide, such as 2′-OMe modified ribose (RNA), 2′-O-ethyl modified ribose, 2′-O-propyl modified ribose, and/or substituted derivatives of these modifications such as halogenated derivatives. An effective AON according to the invention comprises a 2′-OMe ribose and/or a 2′-MOE ribose with a (preferably full) phosphorothioated backbone.

It will also be understood by a skilled person that different AONs can be combined for efficiently skipping of exon 3 from CD274 pre-mRNA or in concert, multiple exons. In a preferred embodiment, a combination of at least two AONs are used in a method of the invention, such as 2, 3, 4, or 5 different AONs. Hence, the invention also relates to a composition comprising a set of AONs comprising at least one AON according to the present invention, optionally further comprising AONs as disclosed herein.

An AON of the present invention can be linked to a moiety that enhances uptake of the AON in cells, preferably epithelial cells of the respiratory tract, liver cells, or cancer cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

An exon 3 skipping AON according to the invention preferably contains all ribonucleosides, which are preferably substituted at the 2′ position of the sugar moiety. Uridines in an AON according to the invention may be 5-methyluridine, or just uridine without a 5-methyl group in the base. Similarly, cytidines in an AON according to the invention may be 5-methylcytidine, or just cytidine without a 5-methyl group in the base. An AON according to the invention may contain one of more DNA residues, and/or one or more nucleotide analogues or equivalents, which means that a “U” as displayed in the sequences of the AONs may also be read as a “T” when it is DNA.

It is preferred that an exon 3 skipping AON of the invention comprises one or more residues that are modified to increase nuclease resistance, and/or to increase the affinity of the AON for the target sequence. Therefore, in a preferred embodiment, the AON sequence comprises at least one nucleotide analogue or equivalent, wherein a nucleotide analogue or equivalent is defined as a residue having a modified base, and/or a modified backbone, and/or a non-naturally occurring internucleoside linkage, or a combination of these modifications. Most preferably, all internucleoside linkages are modified to render the oligonucleotide more resistant to breakdown, and all sugar moieties of the nucleosides are substituted at the 2′, 3′ and/or 5′ position, to render the oligonucleotide more resistant to breakdown. In one embodiment, a nucleotide analogue or equivalent of the invention comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. In one embodiment, the internucleoside linkage is selected from linkers disclosed in WO2009/031091. Particularly preferred are internucleoside linkages that are modified to contain a phosphorothioate. Phosphorothioates are chiral, which means that there are Rp and Sp configurations, known to the person skilled in the art. In a preferred aspect, the chirality of the phosphorothioate linkages is controlled, which means that each of the linkages is either in the Rp or in the Sp configuration, whichever is preferred. The choice of an Rp or Sp configuration at a specified linkage position may depend on the target sequence and the efficiency of binding and induction of providing CD274 exon 3 skipping. However, if such is not specifically desired, a composition may comprise AONs as active compounds with both Rp and Sp configurations at a certain specified linkage position. Mixtures of such AONs are also feasible, wherein certain positions have preferably either one of the configurations, while for other positions such does not matter. In another embodiment, a nucleotide analogue or equivalent of the invention comprises one or more sugar moieties that are mono- or di-substituted at the 2′, 3′ and/or 5′ position such as:
—OH;
—F;
substituted or unsubstituted, linear or branched lower (C1-C10) alkyl, alkenyl, alkynyl, alkaryl, allyl, or aralkyl, that may be interrupted by one or more heteroatoms;
—O-, S-, or N-alkyl (e.g. —O-methyl);
—O-, S-, or N-alkenyl;
—O-, S-, or N-alkynyl;
—O-, S-, or N-allyl;
—O-alkyl-O-alkyl,
-methoxy;
-aminopropoxy;
-methoxyethoxy;
-dimethylamino oxyethoxy; and
-dimethylaminoethoxyethoxy.

It is understood by the skilled person that it is not necessary for all positions in an AON to be modified uniformly. In addition, more than one of the analogues or equivalents may be incorporated in a single AON or even at a single position within an AON. In certain embodiments, an AON of the invention has at least two different types of analogues or equivalents. A preferred exon skipping AON according to the invention is a 2′-O-alkyl phosphorothioated AON, such as an AON comprising a 2′-O-methyl (2′-OMe) modified ribose, a 2′-O-ethyl modified ribose, a 2′-O-propyl modified ribose, and/or substituted derivatives of these modifications such as halogenated derivatives. An effective AON according to the invention comprises a 2′-OMe ribose with a (preferably full) phosphorothioated backbone. Another preferred exon skipping AON according to the invention is a 2′-methoxyethoxy (2′-MOE) phosphorothioated antisense oligonucleotide (an AON comprising 2′-MOE modified riboses, and/or substituted derivatives of these modifications such as halogenated derivatives). An effective AON according to the invention comprises a 2′-MOE ribose with a (preferably full) phosphorothioated backbone.

In a preferred embodiment, the nucleotide analogue or equivalent comprises a modified backbone as outlined above. Examples of such backbones are morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery. However, because the morpholino backbone is uncharged, cationic lipids are not effective mediators of morpholino oligonucleotide uptake in cells. It is further preferred that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A nucleotide analogue or equivalent that may be applied comprises a Peptide Nucleic Acid (PNA), having a modified polyamide backbone. PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA molecule contains no charged phosphate groups, PNA-RNA hybrids are usually more stable than RNA-RNA or RNA-DNA hybrids, respectively.

The sugar moiety can be a pyranose or derivative thereof, or a deoxypyranose or derivative thereof, preferably ribose or derivative thereof, or deoxyribose or derivative thereof. A preferred derivatized sugar moiety, and non-naturally occurring chemical modification of the oligonucleotides of the present invention is Locked Nucleic Acid (LNA), in which the 2′-carbon atom is linked to the 3′ or 4′ carbon atom of the sugar ring thereby forming a bicyclic sugar moiety. As outlined in the accompanying examples, the introduction of several LNAs in the AONs of the present invention may increase the efficiency of skipping even further. The preferred number of LNAs within an AON of the present invention is four (as exemplified herein), but an AON of the present invention may comprise 1, 2, 3, 5, 6, 7, 8, 9, 10, or 11 LNAs and may even be completely modified with LNAs. A preferred LNA comprises 2′-O, 4′-C-ethylene-bridged nucleic acid. These substitutions render the nucleotide analogue or equivalent RNase H and nuclease resistant and increase the affinity for the target RNA. Other sugar modified nucleosides include, for example, bicyclohexose nucleic acids (WO2011/017521) or tricyclic nucleic acids (WO2013/154798).

In another embodiment, a nucleotide analogue or equivalent of the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

### AON Features

In one embodiment, an exon skipping molecule as defined herein is an AON that binds and/or is complementary to a specified sequence. Binding to one of the specified target sequences, preferably in the context of the CD274 pre-mRNA may be assessed via techniques known to the skilled person. A preferred technique is a gel mobility shift assay as described in EP1619249. In a preferred embodiment, an exon skipping AON is said to bind to one of the specified sequences as soon as a binding of said molecule to a labeled target sequence is detectable in a gel mobility shift assay.

In all embodiments of the invention, an exon skipping molecule is preferably an AON. Preferably, an exon skipping AON according to the invention is an AON that induces the skip of one or more exons from human CD274 pre-mRNA. Preferably, exon 3 is skipped, but other exons may be co-skipped by using the AON of the present invention, which does not limit its scope. It may be preferred to have multiple exons being skipped to decrease the PD-L1 functionality. A preferred AON of the present invention comprises or consists of a sequence selected from the group consisting of: SEQ ID NO:1, 2, 3, 4, 5, 6, 7, 8, 9, 10, 11, or 12. More preferably, the AON comprises or consists of a sequence selected from the group consisting of: SEQ ID NO: 1, 2, 4, 7, 8, 9, 10, 11, and 12. Even more preferably, the AON comprises or consists of a sequence selected from the group consisting of: SEQ ID NO:1, 7, 9, and 12. Most preferably, the AON comprises or consists of a sequence selected from the group consisting of SEQ ID NO:9 and 12.

The invention provides a method for designing an exon 3 skipping AON able to induce skipping of exon 3 of the human CD274 pre-mRNA. First, said AON is selected to bind to and/or to be complementary to exon 3 and/or its surrounding intron sequences as shown in SEQ ID NO:13, which includes the full exon 3 sequence of the human CD274 gene and part of the upstream and downstream intron sequences. It is to be understood, that although SEQ ID NO:13 and 14 display DNA sequences, these also represent their respective RNA sequences, when transcribed into pre-mRNA and subsequently mRNA. The pre-mRNA is the preferred target molecule for the AONs of the present invention.

In a preferred method at least one of the following aspects has to be taken into account for designing, improving said exon skipping AON further: the exon skipping AON preferably does not contain a CpG island or a stretch of CpG islands; and the exon skipping AON has acceptable RNA binding kinetics and/or thermodynamic properties. The presence of a CpG or a stretch of CpG in an AON is usually associated with an increased immunogenicity of said AON. This increased immunogenicity is undesired since it may induce damage of the tissue to be treated. Immunogenicity may be assessed in an animal model by assessing the presence of CD4+ and/or CD8+ cells and/or inflammatory mononucleocyte infiltration. Immunogenicity may also be assessed in blood of an animal or of a human being treated with an AON of the invention by detecting the presence of a neutralizing antibody and/or an antibody recognizing said AON using a standard immunoassay known to the skilled person. An inflammatory reaction, type I-like interferon production, IL-12 production and/or an increase in immunogenicity may be assessed by detecting the presence or an increasing amount of a neutralizing antibody or an antibody recognizing said AON using a standard immunoassay. The RNA binding kinetics and/or thermodynamic properties are at least in part determined by the melting temperature of an AON (Tm; calculated with an oligonucleotide properties calculator known to the person skilled in the art), and/or the free energy of the AON-target exon complex. If a Tm is too high, the AON is expected to be less specific. An acceptable Tm and free energy depend on the sequence of the AON. Therefore, it is difficult to give preferred ranges for each of these parameters. An acceptable Tm may be ranged between 35 and 70° C. and an acceptable free energy may be ranged between 15 and 45 kcal/mol.

An AON of the invention is preferably one that can exhibit an acceptable level of functional activity. A functional activity of said AON is preferably to induce the skipping of exon 3 from CD274 pre-mRNA (or in other words, to reduce the inclusion of exon 3 in CD274 mRNA) to a certain acceptable level, to provide an individual with a non-functional PD-L1 protein and/or at least in part decreasing the production of a functional PD-L1 protein. In a preferred embodiment, an AON is said to be capable of inducing skipping of CD274 exon 3, when the CD274 exon 3 skipping percentage as measured by real-time quantitative RT-PCR analysis or digital droplet PCR (ddPCR) is at least 2-10%, preferably at least 10-20%, more preferably at least 20-30%, even more preferably at least 30-40%, and most preferably at least 45%, at least 50%, at least 55%, at least 60%, at least 65%, at least 70%, at least 75%, at least 80%, at least 85%, at least 90%, at least 95%, or 100% as compared to a control RNA product not treated with an AON or a negative control AON. The present disclosure now enables the skilled person to generate an AON that provides significant levels of exon 3 skipping from CD274 pre-mRNA. It is to be understood that when AONs become too short (such that they become non-specific for the target sequence), or too long (such that they can no longer enter the cell, aggregate and/or become degraded), even though they are complementary to (a part of) the exon 3 sequences+/−its surrounding sequences, that they would not be considered part of the invention if they are incapable of providing exon 3 skipping from the human CD274 pre-mRNA, with the percentages given above, and as outlined in detail herein.

An AON according to the invention preferably comprises or consists of a sequence that is complementary to part of SEQ ID NO:13 or 14 (or in fact their (pre-) mRNA equivalents) and can induce exon 3 skipping from human CD274 pre-mRNA.

In a preferred embodiment, the length of the complementary part for the AON of the invention is at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. More preferably, the length of said complementarity is 15, 16, 17, 18, 19, 20, 21, 22, 23, 24, 25, 26, 27, 28, 29, or 30 nucleotides. Most preferably, the length of said complementarity is 16, 17, 18, 19, 20, 21, 22, 23, 24, or 25 nucleotides. From an AON side, the preferred length of an AON according to the invention is at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. Preferably, the length of an AON according to the invention is 15, 16, 17, 18, 19, 20, 21, 22, 23, 24, 25, 26, 27, 28, 29, or 30 nucleotides. Most preferably, the length of an AON according to the invention is 16, 17, 18, 19, 20, 21, 22, 23, 24, or 25 nucleotides. Additional flanking sequences may be used to modify the binding of a protein to the AON, or to modify a thermodynamic property of the AON, more preferably to modify target RNA binding affinity. It is thus not absolutely required that all the bases in the region of complementarity are capable of pairing with bases in the opposing strand. For instance, when designing the AON one may want to incorporate for instance a residue that does not base pair with the base on the complementary strand. Mismatching may, to some extent, be allowed, if under the circumstances in the cell, the stretch of nucleotides is sufficiently capable of hybridizing to the complementary part. In this context, ‘sufficiently’ preferably means that in a gel mobility shift assay as noted above, binding of an AON is detectable.

Skipping of targeted exon 3 may be assessed by RT-PCR (such as e.g. described in EP1619249 and WO 2016/005514) or ddPCR. The complementary regions are preferably designed such that, when combined, they are specific for the exon and/or its surrounding sequences in the pre-mRNA. Such specificity may be created with various lengths of complementary regions as this depends on the actual sequences in other (pre-) mRNA molecules in the system. The risk that the AON also will be able to hybridize to one or more other pre-mRNA molecules decreases with increasing size of the AON. It is clear that AONs that mismatch in the region of complementarity but that retain the capacity to hybridize and/or bind to the targeted region(s) in the pre-mRNA, can be used in the invention. However, preferably at least the complementary parts do not mismatch as AONs that do not mismatch in the complementary part typically have a higher efficiency and a higher specificity than AONs that do mismatch in one or more complementary regions. It is thought that higher hybridization strengths (i.e. increasing number of interactions with the opposing strand) are favorable in increasing the efficiency of the process of interfering with the splicing machinery of the system. An exon skipping AON of the invention, when manufactured, is preferably an isolated single stranded antisense molecule in the absence of its (target) counterpart sequence.

It will also be understood by a skilled person that different AONs can be combined for efficiently skipping of CD274 exon 3. In a preferred embodiment, a combination of at least two AONs are used in a method of the invention, such as 2, 3, 4, or 5 different AONs. Hence, the invention also relates to a set of AONs comprising at least one AON according to the present invention. Nevertheless, from a regulatory and ease-of-production point of view, it is preferred that the medicament only comprises a single AON of the present invention.

An AON can be linked to a moiety that enhances uptake of the AON in cells, such as liver cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain. Asialoglycoprotein receptor (ASGPr) mediated delivery is particularly useful for targeting hepatocytes in liver. Oligonucleotide conjugates comprising the oligonucleotide and asialoglycoprotein receptor targeting conjugate moiety have been successful in targeting liver hepatocytes (Ostergaard et al. 2005, Bioconjug Chem 26(8):1451-1455; Huang 2017, Mol Ther Nucleic Acids 6:116-132). The receptor targeting conjugate moiety can be at least one tri-valent N-acetylgalactosamine (GalNAc) moiety. The conjugation moiety and the oligonucleotide may be linked together by a biocleavable linker from the 3′- or 5′-end of the oligonucleotide. Another alternative might be using nanocarrier formulations that allow intact oligo distribution in hepatocytes.

An exon 3 skipping AON according to the invention may be indirectly administrated using suitable means known in the art. It may for example be provided to an individual or a cell, (cancerous) tissue or organ of said individual as is (in naked and/or isolated form), or in the form of an expression vector wherein the expression vector encodes a transcript comprising said oligonucleotide. The expression vector is preferably introduced into a cell, (cancerous) tissue, organ or individual via a gene delivery vehicle. In a preferred embodiment, there is provided a viral-based expression vector comprising an expression cassette or a transcription cassette that drives expression or transcription of an AON as identified herein. Accordingly, the invention provides a viral vector expressing a CD274 exon 3 skipping AON according to the invention when placed under conditions conducive to expression of the exon skipping AON. A cell can be provided with an exon skipping molecule capable of interfering with essential sequences that result in highly efficient skipping of exon 3 from the CD274 pre-mRNA by plasmid-derived AON expression or viral expression provided by adenovirus- or adeno-associated virus-based vectors. Expression may be driven by a polymerase II-promoter (Pol II) such as a U7 promoter or a polymerase III (Pol III) promoter, such as a U6 RNA promoter. A preferred delivery vehicle is AAV, or a retroviral vector such as a lentivirus vector and the like. Also, plasmids, artificial chromosomes, plasmids usable for targeted homologous recombination and integration in the human genome of cells may be suitably applied for delivery of an oligonucleotide as defined herein. Preferred for the current invention are those vectors wherein transcription is driven from Pol III promoters, and/or wherein transcripts are in the form fusions with U1 or U7 transcripts, which yield good results for delivering small transcripts. It is within the skill of the artisan to design suitable transcripts. Preferred are Pol III driven transcripts, preferably, in the form of a fusion transcript with an U1 or U7 transcript, known to the person skilled in the art.

Typically, when the exon 3 skipping AON is delivered by a viral vector, it is in the form of an RNA transcript that comprises the sequence of an oligonucleotide according to the invention in a part of the transcript. An AAV vector according to the invention is a recombinant AAV vector and refers to an AAV vector comprising part of an AAV genome comprising an encoded exon 3 skipping AON according to the invention encapsidated in a protein shell of capsid protein derived from an AAV serotype. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an AAV serotype such as AAV1, 2, 3, 4, 5, 6, 7, 8, 9 and others. A protein shell may also be named a capsid protein shell. AAV vector may have one or preferably all wild type AAV genes deleted but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV2 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention. Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector. More preferably, a recombinant AAV vector according to the invention comprises a capsid protein shell of AAV serotype 2 and the AAV genome or ITRs present in said vector are derived from AAV serotype 8; such vector is referred to as an AAV 2/8 vector. More preferably, a recombinant AAV vector according to the invention comprises a capsid protein shell of AAV serotype 2 and the AAV genome or ITRs present in said vector are derived from AAV serotype 9; such vector is referred to as an AAV 2/9 vector. More preferably, a recombinant AAV vector according to the invention comprises a capsid protein shell of AAV serotype 2 and the AAV genome or ITRs present in said vector are derived from AAV serotype 2; such vector is referred to as an AAV 2/2 vector. A nucleic acid molecule encoding an exon 3 skipping AON according to the invention represented by a nucleic acid sequence of choice is preferably inserted between the AAV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid.

Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in said AAV vector replication and packaging on the other hand. “AAV helper virus” provides additional functions required for AAV replication and packaging.

Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456. Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art. A preferred AAV vector according to the invention is an AAV vector, preferably an AAV2/5, AAV2/8, AAV2/9 or AAV2/2 vector, expressing an CD274 exon 3 skipping AON according to the invention that comprises or consists of a sequence that is complementary or substantially complementary to a nucleotide sequence as shown in SEQ ID NO:13 or 14.

An exon 3 skipping AON according to the invention can be delivered as is (i.e. naked and/or in isolated form) to an individual, a cell, (cancerous) tissue or organ of said individual. When administering an exon 3 skipping AON according to the invention, it is preferred that the AON is dissolved in a solution that is compatible with the delivery method. Such delivery to respiratory tract cells or liver cells or other relevant cells may be in vivo, in vitro or ex vivo. Nanoparticles and micro particles that may be used for in vivo AON delivery are well known in the art. Alternatively, a plasmid can be provided by transfection using known transfection reagents. For intravenous, subcutaneous, intramuscular, intrathecal and/or intraventricular administration it is preferred that the solution is a physiological salt solution. Particularly preferred in the invention is the use of an excipient or transfection reagents that will aid in delivery of each of the constituents as defined herein to a cell and/or into a cell (preferably a liver cell). Preferred are excipients or transfection reagents capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection reagents comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), Lipofectin™, DOTAP and/or viral capsid proteins that are capable of self-assembly into particles that can deliver each constituent as defined herein to a cell, preferably a liver cell. Such excipients have been shown to efficiently deliver an AON to a wide variety of cultured cells, including liver cells. Their high transfection potential is combined with an excepted low to moderate toxicity in terms of overall cell survival. The ease of structural modification can be used to allow further modifications and the analysis of their further (in vivo) nucleic acid transfer characteristics and toxicity. Lipofectin represents an example of a liposomal transfection agent. It consists of two lipid components, a cationic lipid N-[1-(2,3 dioleoyloxy)propyl]-N, N, N-trimethylammonium chloride (DOTMA) (cp. DOTAP which is the methylsulfate salt) and a neutral lipid dioleoylphosphatidyl ethanolamine (DOPE). The neutral component mediates the intracellular release. Another group of delivery system are polymeric nanoparticles. Polycations such as diethylamino ethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver AONs across cell membranes into cells. In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an AON. The skilled person may select and adapt any of the above or other commercially available alternative excipients and delivery systems to package and deliver an exon skipping molecule for use in the current invention to deliver it for immunotherapy.

An exon 3 skipping AON according to the invention could be covalently or non-covalently linked to a targeting ligand specifically designed to facilitate the uptake into the cell, cytoplasm and/or its nucleus. Such ligand could comprise (i) a compound (including but not limited to peptide(-like) structures) recognizing cell, (cancerous) tissue or organ specific elements facilitating cellular uptake and/or (ii) a chemical compound able to facilitate the uptake in to cells and/or the intracellular release of an oligonucleotide from vesicles, e.g. endosomes or lysosomes. Therefore, in a preferred embodiment, an exon 3 skipping AON according to the invention is formulated in a composition or a medicament or a composition, which is provided with at least an excipient and/or a targeting ligand for delivery and/or a delivery device thereof to a cell and/or enhancing its intracellular delivery. In a particularly preferred embodiment, the AON of the present invention is conjugated to at least one asialoglycoprotein receptor targeting conjugate moiety, such as a conjugate moiety comprising at least one N-Acetylgalactosamine (GalNAc) moiety, for instance for delivery to liver cells and/or for the treatment of cancer of the liver cells, or (chronic) infections of the liver, such as in the case of hepatitis infections. The conjugation moiety and the AON may be linked together by a linker, preferably a biocleavable linker.

It is to be understood that if a composition comprises an additional constituent such as an adjunct compound as later defined herein, each constituent of the composition may not be formulated in one single combination or composition or preparation. Depending on their identity, the skilled person will know which type of formulation is the most appropriate for each constituent as defined herein. In a preferred embodiment, the invention provides a composition or a preparation which is in the form of a kit of parts comprising an exon 3 skipping AON according to the invention and a further adjunct compound as later defined herein. If required, an exon 3 skipping AON according to the invention or a vector, preferably a viral vector, expressing an exon 3 skipping AON according to the invention can be incorporated into a pharmaceutically active mixture by adding a pharmaceutically acceptable carrier. Accordingly, the invention also provides a composition, preferably a pharmaceutical composition, comprising an exon 3 skipping AON according to the invention, or a viral vector according to the invention and a pharmaceutically acceptable excipient. Such composition may comprise a single exon 3 skipping AON or viral vector according to the invention, but may also comprise multiple, distinct exon 3 skipping AON or viral vectors according to the invention. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington (Remington. 2000. The Science and Practice of Pharmacy, 20th Edition. Baltimore, Md.: Lippincott Williams Wilkins). Each feature of said composition has earlier been defined herein.

### EXAMPLES

### Example 1: Use of Antisense Oligonucleotides to Skip Exon 3 from CD274 Pre-mRNA in Human Hepatocellular Carcinoma Cells

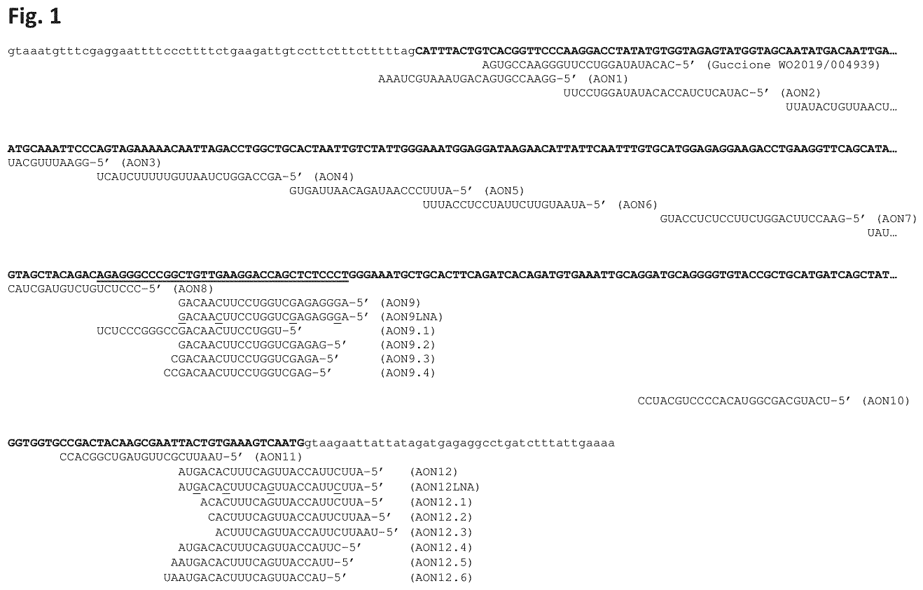

In view of the fact that exon 3 of the human CD274 gene is relatively small (342 nt), the inventors initially designed twelve antisense oligonucleotides (AONs 1 to 12; SEQ ID NO:1-12) that covered most of exon 3, in which AON1 is partly complementary to the upstream intron and AON12 is partly complementary to the downstream intron. FIG. 1 shows the twelve AONs opposite to their respective target regions (AON1 to AON12), together with an AON specifically discussed in the art (Guccione; SEQ ID NO:15). The design was, amongst others, based on GC content and Tm, for proper interaction with the target sequence. A negative control AON that was used in the skipping experiments had the following sequence: 5′-UUCUCAGGAAUUUGUGUCUUU-3′ (SEQ ID NO:16). All CD274 specific AONs were fully phosphorothioated in the inter-nucleoside linkages and all riboses were substituted at the 2′ position with 2′-O-methyl (2′-OMe). The control AON was substituted at the 2′ position with 2′-O-methoxyethyl (2′-methoxyethoxy; 2′-MOE) and all its inter-nucleoside linkages were phosphorothioated.

Human hepatocellular carcinoma cells (HepG2; ATCC-HB-8065) were cultured in ATCC-EMEM medium with 10% FBS and pen/strep and seeded at 1×10⁵ cells/well in a 12-well standard culture dish before transfection. After 24 h cells were treated with IFN-γ (Gibco) to a final concentration of 1 ng/mL (volume well=1 mL) and incubated for 8 h to induce the expression of endogenous PD-L1. Then, cells were transfected with AONs to a concentration of 150 nM using Lipofectamine 2000 (Invitrogen) using the protocol provided by the manufacturer and using general methods known to the person skilled in the art. After 6.5 h cells were lysed and RNA was isolated using the Promega RNA isolation kit (Cat# AM9937) applying the protocol provided by the manufacturer. Subsequently, cDNA was synthesized using the Thermo Fisher Maxima RT kit (Cat #K1652) using the protocol provided by the manufacturer, followed by a PCR with 35 cycles using the following primer set: upstream primer 5′-GCAGGGCATTCCAGAAAGAT-3′ (SEQ ID NO:17)+downstream primer 5′-ACATCCATCATTCTCCCTTTTCT-3′ (SEQ ID NO:18) using methods known to the person skilled in the art. The PCR product of a wild type sequence would render a PCR product of 824 nt, and if exon 3 would be skipped, a PCR product would be 482 nt in length. PCR products were loaded and analyzed on a Bioanalyzer using the DNA1000 kit (Cat#5067-1504), following the protocol provided by the manufacturer.

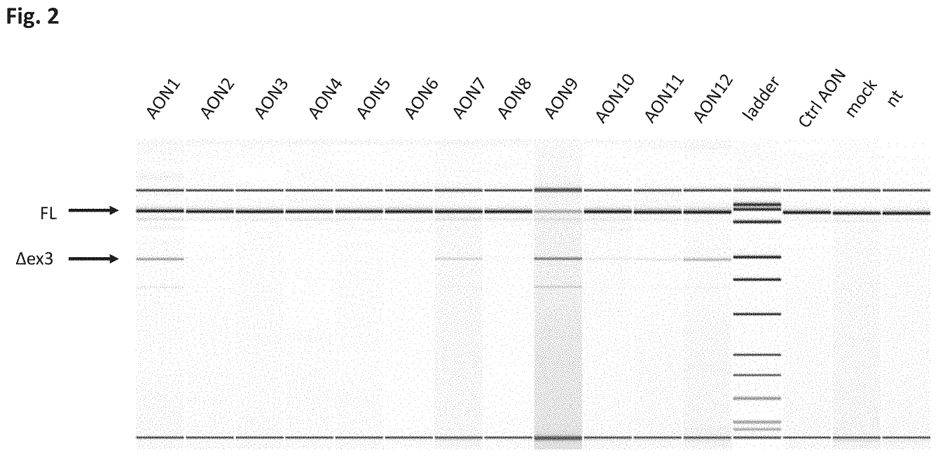

Results of the experiment described above are shown in FIG. 2. The full-length PCR product (824 nt) is indicated by the upper arrow (FL), whereas the PCR product from mRNA from which exon 3 is skipped (482 nt) is indicated by the lower arrow (Δex3). The lower band (+/−367 nt) was investigated and found to be an RNA product from which exon 3 was fully skipped and from which, in addition, a part of exon 4 was skipped. The additional skipped part from exon 4 that was identified (after sequencing; data not shown) appeared to have the following sequence:

(SEQ ID NO: 19)
5′-TAAGACCACCACCACCAATTCCAAGAGAGAGGAGAAGCTTTTCAA
TGTGACCAGCACACTGAGAATCAACACAACAACTAATGAGATTTTCTA
CTGCACTTTTAGG-3′.

Proper skipping results were obtained with AON1, AON7, AON9, and AON12, with AON9 and AON12 performing best. Some skipping could be detected with AON2, AON4, AON8, AON10, and AON11, and no skipping could be detected with AON3, AON5, and AON6. No smaller PCR products were detected with the negative controls (mock, nt and ctrl AON). The 482 nt lower band of the Bioanalyzer was excised and used for sequencing, which showed that the 5′ exon 4 sequence was preceded directly by the 3′ sequence of exon 2 (sequence data not shown). These results together show that the inventors of the present invention were able—for the first time—to obtain exon 3 skipping from human CD274 pre-mRNA in human hepatocellular carcinoma cells, using chemically modified AONs on an endogenous target.

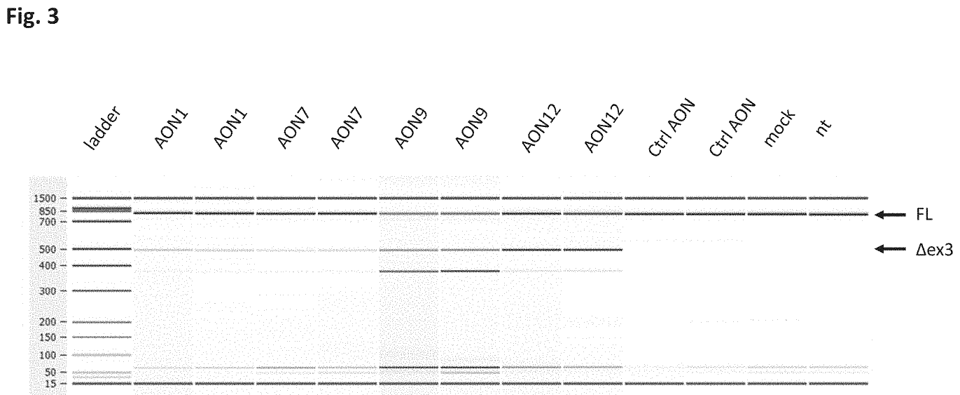

The experiment above was repeated, using the same cells, in the same seeding-, IFN-γ stimulation- and transfection set up, using the best performing AONs 1, 7, 9 and 12, in duplicates. Results (FIG. 3) show that, again, the inventors were able to obtain exon 3 skipping from human CD274 pre-mRNA with AON9 and AON12 that were outperforming AON1 and AON7. Notably, transfection of AON9 resulted in the smaller product (with the full exon 3 skip and the partial exon 4 skip), whereas AON12 predominantly gave the 482 nt product representing the exon 3-only skip. This shows that, depending on the position of complementarity in the CD274 pre-mRNA target sequence, very efficient exon 3 skipping can be obtained, in any case with AON9 and with AON12, and that targeting an internal exon 3 sequence (AON9) and targeting the exon 3 boundary together with its downstream intron (AON12) results in a high efficient exon 3 skip.

### Example 2: Comparison of Best Performing Exon 3 Skipping AONs with an Oligonucleotide from the Art

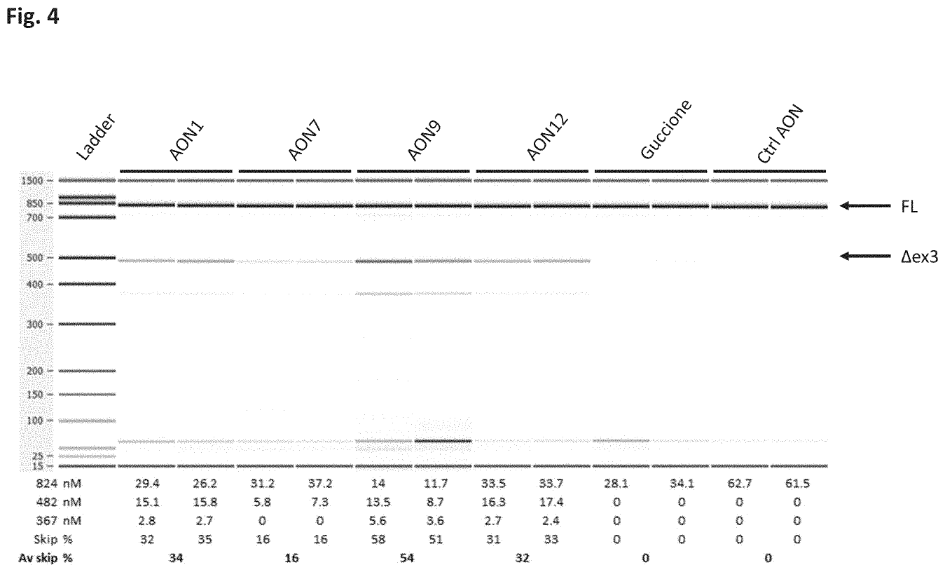

WO 2019/004939 discloses an AON (0915_318_20 M_E3; page 9, Table 2; SEQ ID NO:19411 therein; SEQ ID NO:15 herein; see also FIG. 1) presumably designed for the skip of exon 3 from PD-L1. Notably, whereas WO 2019/004939 discloses splice switching effects on PD-1 and CTLA4 using AONs, downregulation of PRF protein expression using AONs, and exon skipping of CD244, TIM3, TGIT, PRDM1, REL, CD160, and CD80 RNA target molecules using AONs, it completely fails to show exon 3 and exon 4 skipping data on CD274 pre-mRNA. The inventors of the present invention, while obtaining proper exon 3 skipping in CD274 pre-mRNA, especially with AON1, AON7, AON9 and AON12 (see Example 1), were interested to see how their results would compare to an exon skip using the specific exon 3 targeting AON from the prior art (herein referred to as the ‘Guccione’ AON). For this, an identical transfection and RT-PCR experiment as outlined above in Example 1 was performed, but now in IFN-γ induced human HeLa cells, using AON1, AON7, AON9, AON12 and the Guccione AON that were all fully modified with 2′-OMe modifications. RT-PCR procedures and primers were as described above. The Guccione AON was apparently 2′-OMe modified in WO 2019/004939 and therefore in that particular modified form manufactured for the experiment described here. Results are shown in FIG. 4. Clearly, the Guccione AON did not result in any significant exon 3 skip from human CD274 pre-mRNA (even calculated to be 0% based on the Bioanalyzer results, although a very faint band could be seen at the Δex3 level), whereas AON1, AON7, AON9 and AON12 again gave very high exon skip percentages (up to 54% skip, averaged), with again AON9 outperforming the other AONs. It was concluded that the inventors of the present invention were the first to achieve, and to show exon 3 skipping from human CD274 pre-mRNA using an antisense oligonucleotide targeting the exon 3 target RNA within the CD274 pre-mRNA. In the hands of the inventors of the present invention, the single AON known from the prior art did not provide significant exon 3 skip.

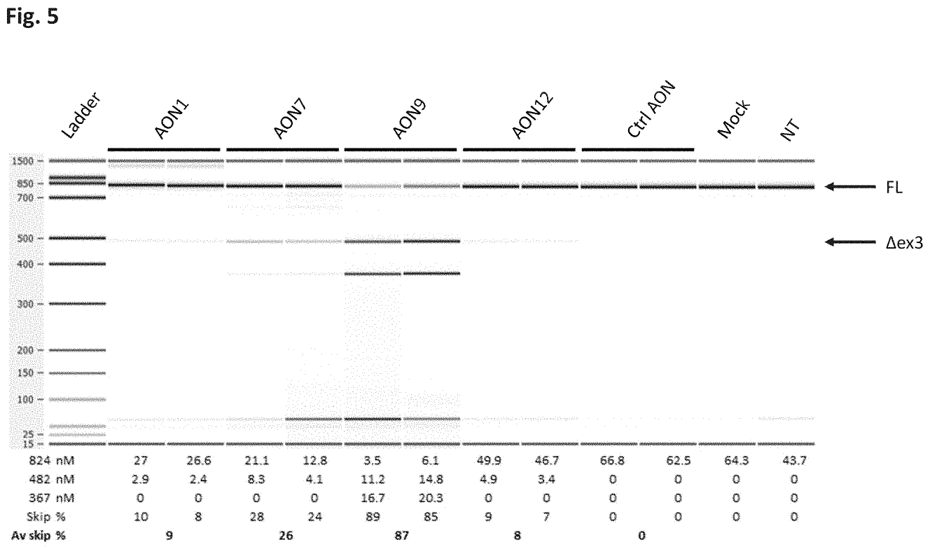

In a second experiment using HeLa cells, this experiment was repeated with AON1, AON7, AON9, and AON12. A non-targeting AON served as a negative control (Ctrl AON), while a mock transfection, and no transfection served as additional negative controls. In this particular case, AON1, AON7, AON9, AON12 and the control AON were all fully modified with 2′-MOE. No such version of the Guccione AON was produced (as it was also not disclosed in WO 2019/004939). The results of this experiment are shown in FIG. 5, and clearly indicate that AON9 in this 2′-MOE modified version was able to achieve almost 90% skipping efficiency. AON12 on the other hand performed less efficient in the 2′-MOE version in comparison to the results obtained with its 2′-OMe modified version (see FIG. 4). It may be that depending on the target sequence in the target RNA molecule, either 2′-OMe or 2′-MOE, or an AON in which 2′-OMe and 2′-MOE modifications are both present and located at specified positions may give even better skipping efficiencies.

### Example 3: Optimization of Best Performing Exon 3 Skipping AONs

As stated in the previous examples, AON1, AON7, AON9 and AON12 were the best performing PD-L1 exon 3 skipping oligonucleotides. AON9 and AON12 were subjected to further optimization. Length and binding region were changed thereby potentially altering the binding affinity and splice modulation capabilities. Both 2′-OMe and 2′-MOE variants were tested as well as chimeric LNA substitutions for both chemistries, in which 4 nucleotides were LNA. FIG. 1 shows the optimized additional AONs (AON9LNA, AON9.1, AON9.2, AON9.3, AON9.4, AON12LNA, AON12.1, AON12.2, AON12.3, AON12.4, AON12.5, and AON12.6), with the underlined sequence (SEQ ID NO:20) being the target area for the (optimized) AON9 AONs. The LNAs in the AONs are underlined in FIG. 1. Experiments were performed using the same methods as described above in HeLa cells and analysed through the RNA isolation-cDNA synthesis-Bioanalyzer workflow.

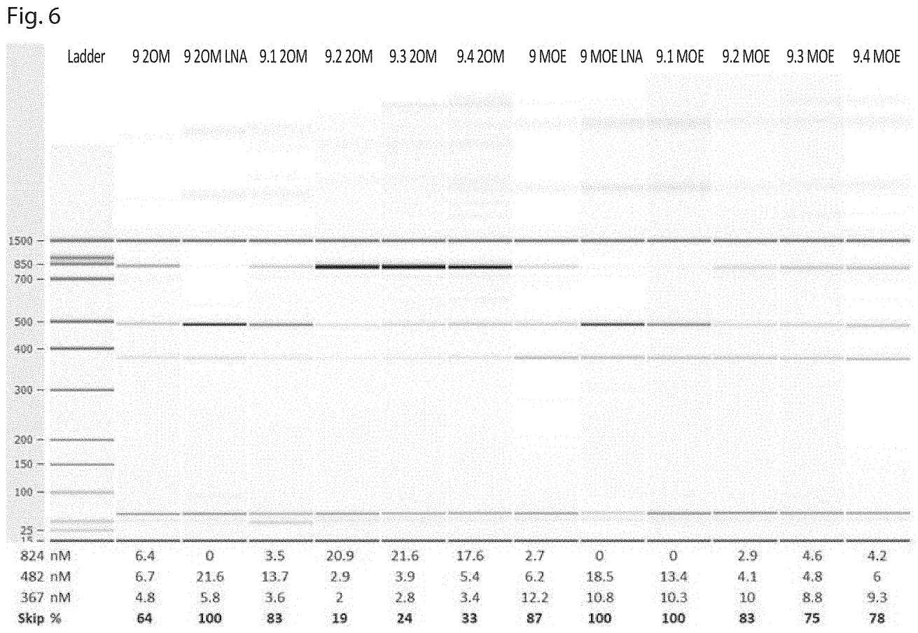

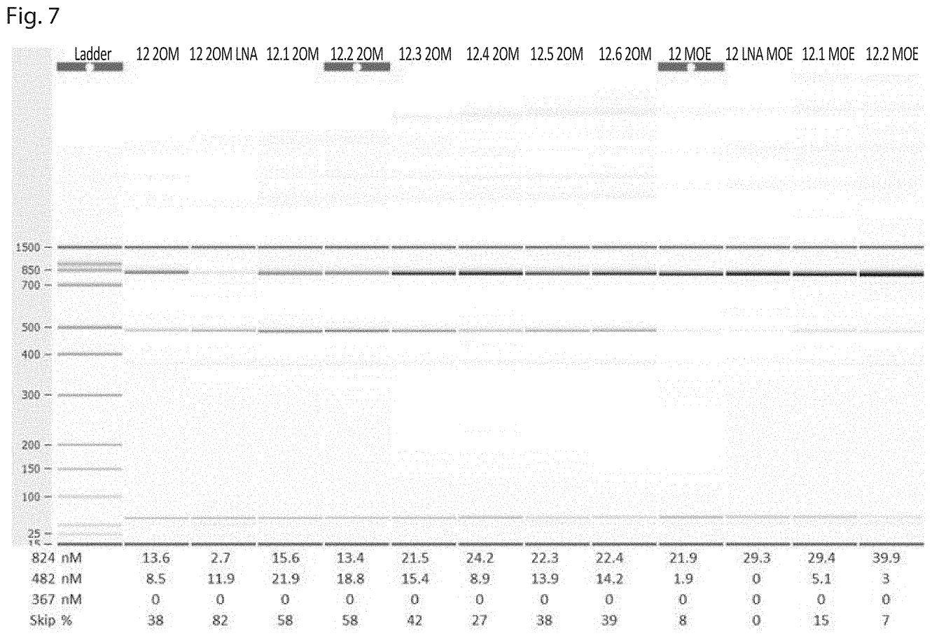

The Bioanalyzer results shown in FIG. 6 clearly indicate an increased skipping efficiency of AON9.1 (shifted region of complementarity) and AON9LNA (in which 4 nucleotides are LNA) for both 2′-OMe and 2′-MOE chemistries when compared to the original AON9. Up to 100% skip efficiency was observed. AON12.1, AON12.2 and the AON12LNA chimeric oligonucleotide showed improved skipping capabilities compared to the original AON12, but only for the 2′-OMe variants (FIG. 7). More than 80% skip was achieved using the 2′-OMe AON12LNA chimeric. This indicates that the inventors were able to obtain exon 3 skipping from human CD274 pre-mRNA with AON9 and AON12, and that such could be further improved by additional chemical modifications of the AON. Also, the inventors show that very efficient exon 3 skipping can be obtained and improved based on position of complementarity in the CD274 pre-mRNA target sequence and AON chemistry.

### Example 4: T Cell Proliferation/Apoptosis Assay

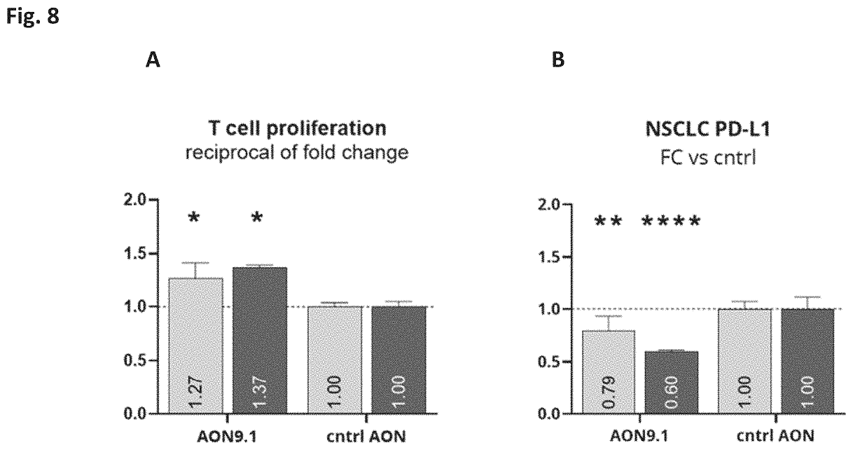

Mature T cells ideally only recognize foreign antigens combined with self-Major Histocompatibility Complex (MHC) molecules in order to mount an appropriate immune response. Cancerous or specific virus-infected cells act as antigen presenting cells (APC) and activate T-cells through T cell receptor (TCR)-Antigen-MHC interaction. However, secondary inhibitory and stimulatory receptor ligand “checkpoints” are needed to halt or unleash a proper immune response, respectively. The transmembrane proteins PD1 and PD-L1 are known to regulate immune responses through cell-to-cell interactions. PD1 (receptor)/PDL-1 (ligand) signaling results in dampened and lowered T cell response and to improper immune surveillance. Where T cell activation and stimulation normally leads to increased T cell proliferation and survival, PD1 signaling is believed to inhibit this process and induces apoptosis and exhaustion. In theory, cell surface PD-L1 reduction via oligonucleotide interference could affect T cell proliferation and/or apoptosis state when APC and T-cells are co-cultured. Proliferation and apoptosis can be measured using a fluorescent acquired cell sorter (FACS). Cell proliferation is commonly determined using a cell membrane crossing fluorescent dye (e.g. Cell Trace Violet). With each cell division fluorescent intensity per cell is halved and measured as Median Fluorescent Intensity (MFI) using FACS. Blocking or inhibiting immune checkpoints could influence T cell apoptosis and visualized on FACS through Annexin/PI staining. An in vitro APC T-cell co-culture model system (T-cell apoptosis/proliferation assay) is widespread regarded as a proper method to test immune checkpoint functionality. It was envisioned by the inventors that AON induced PD-L1 exon 3 skip inhibits PD1/PD-L1 signaling which would then result in decreased T cell apoptosis and increased proliferation. The proliferation method on wild type T cells was generally performed as follows, whereas the apoptosis assay is generally performed along similar lines. At timepoint—24 hr, non-small cell lung cancer cells (NSCLC cells) were seeded. At t=0 hr transfection with 150 nM AON9.1 (and a control oligonucleotide) was performed as described in Example 1. At t=24 hr the transfection medium was replaced with normal fresh medium without oligonucleotides. At t=48 hr, isolation of healthy donor PBMC derived T-cells was performed according to the protocol of the isolation kit manufacturer (MACS, Cat. No. 130-050-101). After maintaining the cells in culture medium, cells were spun down, the medium was aspirated, and cells were resuspended in 240 μl MACS buffer and 60 μl MACS CD3 microbeads. The cell bead suspension was left at 4° C. for 20 min to allow proper binding. Fluorescent labelling was performed according to the manufacturers protocol (ThermoFisher Scientific, Cat. No. C34557). PD-1 expression and proliferation were stimulated using anti-CD3/CD28 beads according to the manufacturers protocol (ThermoFisher Scientific, Cat. No. 11131D) preceding co-culture start. In case of apoptosis experiments, T cells Annexin V/PI staining is also performed according to the protocol of the supplier (Miltenyi kit, Cat. No. 130-092-052). At t=72 hr, the transfected NSCLC and isolated T cells were co-cultured for 48 hr (t=120 hr) and 72 hr (t=144 hr) at which timepoints fluorescence was detected and proliferation was determined. After co-incubation of the cells, co-culture supernatants containing suspension cells (mainly T cells) were collected in capped FACS tubes. Tubes were centrifuged at 300×g for 10 min and supernatant was aspirated. Cells were washed in 1 mL of 1× Binding Buffer and centrifuged at 300×g for 10 min. Supernatant was aspirated completely. An Annexin V FITC premix was made for 20 samples as follows: 200 μl Annexin V FITC was mixed with 2000 μl 1× Binding Buffer. 110 μl of Annexin V premix was added to all tubes except the controls. This was mixed thoroughly and incubated for 15 min in the dark at RT. Cells were washed by adding 1 mL of 1× Binding Buffer per 10⁶ cells and centrifuged at 300×g for 10 min. Supernatant was aspirated completely. PI 1× in 1× Binding buffer was prepared for twenty staining procedures as follows: 30 μl PI solution was added to 2970 μl 1× Binding Buffer and mixed. Cells were resuspended in 150 μl diluted PI and incubated for at least 5 min. CD3+ stain was performed according to the protocol of the supplier (Miltenyi Biotec, Cat. No 130-113-135), and proliferation was determined with a flow cytometer within 4 hr. Resulting data was analyzed using FlowJo 10. The results obtained after co-culturing T cells with AON9.1-transfected NSCLC's are depicted in FIG. 8A. Because fluorescence diminishes with increased proliferation, the results are depicted ‘reciprocally’ (1/fold change), which then shows that proliferation, in comparison to the cells transfected with control oligonucleotide is increased. The grey bars show the fold increase after 48 hr co-culture, while the dark bars show the fold increase after 72 hr co-culture. The T cells express PD1, whereas the NSCLC cells express PD-L1, which should be lowered after exon 3 skipping of the CD274 pre-mRNA. Since the interaction of PD-L1 on the NSCLC cells with PD1 on the T cells is diminished, T cell proliferation is increased, which indicates that suppression of PD-L1 expression by skipping exon 3 results in an increased T-cell activity.

It was also investigated whether the expression of PD-L1 on the transfected NSCLC cells was indeed lower. For this, from the experiment described above, transfected cells (at the timepoints t=120 and 144 hr) were harvested by aspiration of the culture medium, washed with PBS and incubated for 30 min with Versene (1:5000; Gibco; Cat. No. 15040-033). Subsequently, PD-L1 labelling was performed by incubation with anti-PD-L1 antibody (100 μl 1:20 diluted in FACS buffer; anti-Hu-CD274; Invitrogen; Cat. No. 12598342; FACS buffer from Sigma). The results are shown in FIG. 8B and clearly indicate that the expression of PD-L1 is significantly reduced on the transfected NSCLC cells, supporting the finding that, when co-cultured with T cells, proliferation of T cells is increased. These results strongly suggest that the inventors of the present invention were able, by skipping exon 3 from human CD274 pre-mRNA were able to lower the expression of PD-L1 and therethrough increase the proliferation of T cells, showing the feasibility of this approach in a clinical setting wherein T cell proliferation should be increased and T cell exhaustion should be lowered.

### Example 5: Use of CD274 Exon 3 Skipping Oligonucleotides in the Treatment of Viral Infections of the Respiratory Tract

As outlined herein, in December 2019 a novel coronavirus was reported in Wuhan, China, which was named SARS-CoV-2 causing COVID-19, resulting in a pandemic in the months that followed with almost four hundred thousand casualties and fourteen thousand deaths at the end of March 2020, worldwide. Scientific publications revealed that one of the features that occurred in COVID-19 patients was T cell exhaustion, limiting the clearance of viruses from the infected subject, and being one of the major causes of severe progression of the disease. The inventors of the present invention realized that preventing T cell exhaustion may be instrumental in the treatment of COVID-19 patients by providing an antisense oligonucleotide (AON) to skip exon 3 from CD274 pre-mRNA, because the protein product of the CD274 gene, PD-L1 is a major player in the process of T cell exhaustion. By downmodulating the function of PD-L1, by skipping exon 3 from its pre-mRNA, the protein should no longer interact with its natural receptor PD-1, and thereby no longer induce T cell exhaustion of T cells that migrate to the infection site. Subsequently, this results in a more robust immune response to the coronavirus infection. Hence, the inventors of the present invention contemplated testing this in an animal model as outlined below.

Oligonucleotide AON9 (SEQ ID NO:9) is 100% complementary to its targeting sequence in Homo sapiens, as shown in FIG. 1, but is also 100% complementary to the equivalent target sequence in exon 3 of CD274 in Macaca fascicularis (crab-eating macaque or cynomolgus monkey), which is a good animal model for viral infections of the respiratory tract. Exon 3 of CD274 in the two species share a homology of 97% (data not shown). AON9 is tested for its ability to skip exon 3 in airway epithelial cells in an in vivo setup using such macaque monkeys, followed by an assessment on whether such exon 3 skipping provides a more robust immune response, as well as a more rapid viral clearance upon a challenge with an influenza virus or a coronavirus in the treated monkeys (before or after administration of the oligonucleotide). A variety of administration routes are selected (subcutaneous injection and/or inhalation/nebulization). Inhalation results in a direct delivery of the AON to the respiratory tract and is the preferred route of administration. Subsequently, qualitative and quantitative assessments of mRNA level exon 3 skip are performed. Besides a viral challenge and the response to that challenge in the AON-treated as well as in the control animals (receiving either PBS or a scrambled control oligonucleotide), T cell expansion, viral clearance, disease symptoms, general well-being, prolonged survival and time of recovery are monitored.

1. An antisense oligonucleotide (AON) that is capable of inducing skipping at least exon 3 from CD274 pre-mRNA, wherein the AON comprises a sequence:
that is substantially complementary to a sequence that is entirely within exon 3 of the CD274 gene;
that is substantially complementary to a sequence of exon 3 of the CD274 gene and is substantially complementary to a sequence of the intron located upstream of exon 3, and thereby overlaps with the 5′ intron/exon boundary; or
that is substantially complementary to a sequence of exon 3 of the CD274 gene and is substantially complementary to a sequence of the intron located downstream of exon 3, and thereby overlaps with the 3′ exon/intron boundary.

2. The AON according to claim 1, wherein the AON comprises less than 26 nucleotides, preferably wherein the AON consists of 16, 17, 18, 19, 20, 21, 22, 23, 24, or 25 nucleotides.

3. The AON according to claim 1, wherein the AON comprises or consists of a sequence selected from the group consisting of: SEQ ID NO: 1, 2, 4, 7, 8, 9, 10, 11, and 12.

4. The AON according to claim 2, wherein the AON is 100% complementary to a consecutive stretch of nucleotides within the sequence of SEQ ID NO:20.

5. The AON according to any one of claims 1 to 4, wherein the AON comprises at least one non-naturally occurring chemical modification.

6. The AON according to claim 5, wherein the non-naturally occurring modification comprises at least one internucleoside modified linkage, preferably a phosphorothioate modified linkage.

7. The AON according to any one of claims 1 to 6, wherein the AON comprises one or more sugar moieties that is mono- or di-substituted at the 2′, 3′ and/or 5′ position, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

8. The AON according to claim 5, wherein the at least one non-naturally occurring chemical modification is a Locked Nucleic Acid (LNA) modification, preferably wherein 1, 2, 3, or 4 nucleotides are LNA.

9. The AON according to claim 7, wherein the AON comprises at least one sugar moiety carrying a 2′-O-methyl modification and/or wherein the AON comprises at least one sugar moiety carrying a 2′-methoxyethoxy modification.

10. A pharmaceutical composition comprising an AON according to any one of claims 1 to 9, and a pharmaceutically acceptable carrier.

11. A viral vector expressing an AON according to any one of claims 1 to 4.

12. An AON according to any one of claims 1 to 9, a pharmaceutical composition according to claim 10, or a viral vector according to claim 11, for use in the treatment of a chronic or acute viral infection, an (auto-) immune disease or a cancer.

13. The AON for use in the treatment of a chronic or acute viral infection according to claim 12, wherein the viral infection is a respiratory tract infection or a liver infection.

14. The AON for use in the treatment of a liver infection according to claim 13, wherein the infection is caused by HBV or HCV.

15. The AON for use in the treatment of an acute respiratory tract infection according to claim 13, wherein the infection is caused by an influenza virus or a coronavirus.

16. The AON for use in the treatment of an acute respiratory tract infection according to claim 15, wherein the coronavirus is Severe Acute Respiratory Syndrome coronavirus 1 or 2 (SARS-CoV-1 or SARS-CoV-2), or a derivative thereof.

17. A method of inducing skipping of at least exon 3 from CD274 pre-mRNA in a cell, comprising the step of administering to the cell an AON according to any one of claims 1 to 9, a pharmaceutical composition according to claim 10, or a viral vector according to claim 11; optionally further comprising the step of determining whether the skip of exon 3 from the CD274 pre-mRNA has occurred.

18. The method according to claim 16, wherein the cell is an in vitro or ex vivo cultured human cell.

19. The method of claim 16 or 17, wherein the cell is a cell that expresses PD-L1, preferably a cancer cell or a cell that is infected by a virus.

20. A method of treating a human subject suffering from an acute respiratory tract infection caused by a coronavirus, wherein the coronavirus is preferably SARS-CoV-1 or -2, or a derivative thereof, comprising the steps of formulating in a composition an AON according to any one of claims 1 to 9, and administering the formulated AON to the respiratory tract of said subject.

21. A method of modulating the function of PD-L1 in a target cell, comprising the step of administering to the cell an AON according to any one of claims 1 to 9, a pharmaceutical composition according to claim 10, or a viral vector according to claim 11; and allowing the skip of at least exon 3 from the CD274 pre-mRNA that encodes the PD-L1 protein.

22. Use of an AON according to any one of claims 1 to 9, a pharmaceutical composition according to claim 10, or a viral vector according to claim 11 in the manufacture of a medicament for the treatment, prevention or amelioration of a chronic or acute viral infection, an auto-immune disease, or a cancer.


SEQUENCE LISTING (20 sequences)

SEQ ID NO: 1 - RNA, 24 nt, Artificial sequence
  AON1
ggaaccgugacaguaaaugcuaaa

SEQ ID NO: 2 - RNA, 25 nt, Artificial sequence
  AON2
cauacucuaccacauauagguccuu

SEQ ID NO: 3 - RNA, 25 nt, Artificial sequence
  AON3
ggaauuugcauucaauugucauauu

SEQ ID NO: 4 - RNA, 25 nt, Artificial sequence
  AON4
agccaggucuaauuguuuuucuacu

SEQ ID NO: 5 - RNA, 22 nt, Artificial sequence
  AON5
auuucccaauagacaauuagug

SEQ ID NO: 6 - RNA, 22 nt, Artificial sequence
  AON6
auaauguucuuauccuccauuu

SEQ ID NO: 7 - RNA, 25 nt, Artificial sequence
  AON7
gaaccuucaggucuuccucuccaug

SEQ ID NO: 8 - RNA, 21 nt, Artificial sequence
  AON8
cccucugucuguagcuacuau

SEQ ID NO: 9 - RNA, 23 nt, Artificial sequence
  AON9
agggagagcugguccuucaacag

SEQ ID NO: 10 - RNA, 26 nt, Artificial sequence
  AON10
ucaugcagcgguacaccccugcaucc

SEQ ID NO: 11 - RNA, 22 nt, Artificial sequence
  AON11
uaauucgcuuguagucggcacc

SEQ ID NO: 12 - RNA, 25 nt, Artificial sequence
  AON12
auucuuaccauugacuuucacagua

SEQ ID NO: 13 - DNA, 439 bp, Homo sapiens
gtaaatgtttcgaggaattttcccttttctgaagattgtccttctttctttttagcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatggtaagaattattatagatgagaggcctgatctttattgaaaa

SEQ ID NO: 14 - DNA, 342 bp, Homo sapiens
catttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatg

SEQ ID NO: 15 - RNA, 26 nt, Artificial sequence
  Guccione AON
cacauauagguccuugggaaccguga

SEQ ID NO: 16 - RNA, 21 nt, Artificial sequence
  Control non-targeting AON
uucucaggaauuugugucuuu

SEQ ID NO: 17 - DNA, 20 bp, Artificial sequence
  Upstream PCR primer
gcagggcattccagaaagat

SEQ ID NO: 18 - DNA, 23 bp, Artificial sequence
  Downstream PCR primer
acatccatcattctcccttttct

SEQ ID NO: 19 - DNA, 106 bp, Homo sapiens
taagaccaccaccaccaattccaagagagaggagaagcttttcaatgtgaccagcacactgagaatcaacacaacaactaatgagattttctactgcacttttagg

SEQ ID NO: 20 - DNA, 34 bp, Homo sapiens
agagggcccggctgttgaaggaccagctctccct